(19) United States
(12) Patent Application Publication
(10) Pub. No.: US 2010/0248229 A1
(43) Pub. Date: Sep. 30, 2010

(54) INVITRO HUMAN EMBRYONIC MODEL AND A METHOD THEREOF

(75) Inventors: Kaushik Dilip Deb, Bangalore (IN); Satish Mahadeo Totey, Bangalore (IN)

Correspondence Address: OLIFF & BERRIDGE, PLC, P.O. BOX 320850, ALEXANDRIA, VA, 22320-4850, US

(73) Assignee: Stempeutics Research Private Limited, Bangalore (IN)

(21) Appl. No.: 12/450,806

(22) PCT Filed: Apr. 11, 2008
(86) PCT No.: PCT/IN2008/000241

§ 371 Date: Oct. 14, 2009

(30) Foreign Application Priority Data
Apr. 20, 2007 (IN) 00855/CHE/2007

(51) Int. Cl. C12N 5/0735; C12Q 1/68; C12N 11/00; C12N 11/10; C12N 11/08

(52) U.S. Cl. 435/6; 435/366; 435/174; 435/177; 435/178; 435/180

(57) ABSTRACT

The present invention relates to the field of stem cells particularly development of a novel human embryonic model using human embryoid bodies obtained from the human embryonic stem cell. The novel human embryonic model disclosed thus can provide a screening assay for determining the toxic activity of the compound and/or drug.

### FIELD OF INVENTION

The present disclosure relates to the field of stem cells particularly development of a novel human embryonic model using human embryoid bodies obtained from the human embryonic stem cell. The novel human embryonic model disclosed thus can provide a screening assay for determining the toxic activity of the compound and/or drug.

### BACKGROUND OF THE INVENTION AND PRIOR ART

Embryonic stem cells (ESCs) have the potential to differentiate into—any kind of tissues that arises from the germ lineages namely: 1) ectoderm 2) endoderm 3) mesoderm and 4) trophectoderm formed during development of the human. Embryoid bodies (EBs) which are produced from the ESCs are known to have a mixed population of the lineages, and therefore resemble an early human embryo.

Pharmaceutical and other industries regularly come up with several new molecules/drugs/formulations for various therapeutic purposes and sometimes for new contraceptives. The effect of these drugs and also environmental pollutants of the water, air, or soil (like fertilizers, or estrogenic compounds in the air) on the developing fetus in a pregnant mother is usually not known. Similarly, infections of the maternal genital tract and also systemic infections of various kinds can lead to abortions through the formation of poor quality embryos, which eventually fail to implant or cause severe birth defects in the fetus (Deb et al., 2004, 2005, 2006, and 2007). The ability to study the underpinning molecular mechanisms and to be able to evaluate the toxic effect of drugs on the developing human fetus (before they enter the market) is very important. Due to the nature of the problem, there are several ethical limitations, which do not permit such studies in pregnant women.

There are several animal tests (on mouse, frogs, and zebra fish) which are used to screen the toxic effect of such molecules/drugs/formulations on the developing fetus. However, most of these animal studies or models do not exactly mimic the process in human.

Embryonic Stem Cell Test (EST)—The effect of chemicals on 3T3 cells and on ES cells, a permanent cell line derived from mouse embryonic stem cells, can be used to predict teratogenic potential; Invittox Protocol number 113 describes a similar assay in mouse.

Nonylphenol and Octylphenol-Induced Apoptosis in Human Embryonic Stem Cells is Related to Fas-Fas Ligand Pathway (2006) Kim S, Kim B, Shim J, Gil J, Yoon Y, Kim J. Toxicological Sciences, doi:10.1093/toxsci/kf1114 shows a study using hESCs and not EBs. The prior teaches away from the present invention.

Mechanisms underpinning Gram-negative bacterial-vaginosis induced birth anomalies are obscure. Ethical issues limit such studies on peri-implantation stage human embryos. Here we have used embryoid bodies (EBs) as an in vitro model to examine the effect of gram-negative bacterial endotoxins/lipopolysaccharides (LPS) on the faithful induction of germ lineages during embryogenesis. In previous studies we have shown that LPS exposure can render the preimplantation embryo or 5 days old blastocyst inefficient for implantation [15]. The role of LPS-inducible cytokine and pluripotency-related DNA-binding-protein HMGB1 was also studied in these EBs.

Human embryonic stem cells (hESCs) have been widely used to understand the molecular mechanisms underpinning human development. These pluripotent cells provide a reliable source for studying differentiation to all the germ layer lineages namely ectoderm, endoderm, mesoderm and trophectoderm lineages [1, 2]. HESCs have been successfully directed towards the formation of different tissues of various lineages [3]. These cells can also be used to produce preimplantation embryo or blastocyst like entities, known as embryoid bodies (EBs) which consist of a differentiated population of cells representing all the germ layers. These EBs therefore, closely mimic a growing embryo which consists of the placental precursors (trophectoderm) and the cells of the embryo proper (ectoderm, endoderm and mesoderm) [4] It is known that ectoderm forms the skin and the nervous system, the mesoderm forms tissues like the cardiomyocytes, bone and blood, and the endoderm forms the liver, lungs and intestine etc of the developing embryo [5].

Gram-negative bacterial infections of the maternal genital tract, known as bacterial vaginosis, can lead to the formation of poor quality embryos, which fail to implant [6]. Subclinical or silent infections of gram-negative bacteria like Chlamydia trachomatis etc. can also cause birth defects with poorly developed tissues and organs of the fetus [7]. Ethical issues limit studies on the molecular mechanisms underlying such pathogenesis in human embryos. Endotoxin, lipopolysaccaharides (LPS) is the main antigenic component of gram negative bacterial cell wall and is regularly shed in the surrounding environment. LPS is known to cause various peri-natal complications [8]. In previous studies we have established the role of various proinflammatory and other LPS inducible cytokines and growth factors like IL-1α, IL-1β, TNF-α and CSF1 during embryo implantation and in subsequent pregnancy loss [9, 10, 11]. However, the molecular events underlying poor fetal development and birth defects during silent infections are not known. We hypothesize that the presence of LPS in the environment of the developing fetus may selectively inhibit the induction of one or more of the lineages during early pregnancy.

As already discussed, the ability to study the underpinning molecular mechanisms and to be able to evaluate the toxic effect of drugs on the developing human fetus (before they enter the market) is very important. However, due to the nature of the problem, there are several ethical limitations, which do not permit such studies in pregnant women. We have used early stage 5 days old EBs to closely mimic the peri-implantation stage of embryonic development (day 4 to 5). The instant invention overcomes the limitations existing in prior art and enables one to study the underpinning molecular mechanisms and evaluate the toxic effect of molecules on the developing fetus.

### OBJECTS OF THE INVENTION

The main object of the present invention is to obtain an in vitro embryonic model comprising spherical smooth-embryoid body (SSE) for determining effect of molecule

Another object of the present invention is to develop an in vitro method for determining effect of molecule on spherical smooth-embryoid body (SSE)

Yet another object of the present invention is to obtain an in vitro embryo implantation model.

Still another object of the present invention is to develop an in vitro method of determining effect of lipopolysaccharide (LPS) on embryoid bodies (EBs).

### STATEMENT OF THE INVENTION

Accordingly, the present invention relates to an in vitro embryonic model comprising spherical smooth-embryoid body (SSE) for determining effect of molecule; an in vitro method for determining effect of molecule on spherical smooth-embryoid body (SSE) comprising acts of: a) exposing the molecule to the SSE, and b) (i) screening for the effect of the exposure on formation and induction of germ lineages; (ii) screening for the effect of the exposure on germ lineages; (iii) screening for the effect of the exposure on implantating embryo; (iv) screening for the effect of the exposure on differentiation into tissue; and (v) screening for cytotoxic effect of the exposure; or any combination(s) thereof; An in vitro embryo implantation model comprising: a) coat of extracellular matrix onto support matrix having well(s); b) layer of endometrial cells onto the extracellular matrix; and c) spherical smooth-embryoid body (SSE) placed into the well to determine effect of molecule; and an in vitro method of determining effect of lipopolysaccharide (LPS) on embryoid bodies (EBs), said method comprising acts of: a) exposing the EBs to the LPS to trigger expression of gene HMGB1 in cytoplasm of the EBs, and b) observing silencing of mesoderm induction and functional differentiation in the EBs.

### BRIEF DESCRIPTION OF ACCOMPANYING DRAWINGS

FIG. 1: Schematic diagram showing the in vitro implantation model.

FIG. 2a & b: Positive effect of a compound on implantation; FIG. 2a indicates a control and FIG. 2b indicates supportive/enhanced attachment of spheroid cavitating/cytic EB as a result of 20 μm Y27632 exposure.

FIGS. 3a & 3b: Negative effect of a compound on implantation; FIG. 3a indicates Control (attached EBs) and FIG. 3b indicates degrading unattached EBs as a result of uvomorulin antibody treatment (UVO treatment) for 48 hrs.

FIG. 4: Phase contrast pictures of HUES-9 colonies and embryoid bodies. Panel (A) shows morphology of undifferentiated HUES-9 colonies growing on mouse feeder. Panels (B) and (C) show morphologies of normal and lipopolysaccharides-treated day 5 embryoid bodies, respectively. Pictures were acquired at 10× magnifications.

FIG. 5: RT-PCR analysis for the expression of pluripotency and ectoderm, endoderm, mesoderm and trophectoderm markers. (A) Pluripotency (OCT4, NANOG and HMGB1); (B) ectoderm (βIII Tubulin); (C) endoderm (GATA4); (D) mesoderm (Brachury, BMP2, ANP, cTnT, ABCG2, GATA2, HAND1, BMP4); and (E) trophectoderm (βhCG) genes in normal HUES9 cells, EBs and LPS-treated EBS. The lineage markers were found to be absent in the HUES9 cells. The normal EBs shoed expression for all the lineage markers. The LPS-treated EBs showed no expression for the mesoderm markers.

FIG. 6: Immunolocalization of SSEA4, Nanog, HMGB1 and Brachury; and induction of osteoblast differentiation. Panels (A), (B) and (C) show immunolocalization of SSEA4, NANOG and HMGB1, respectively. Panel (D) shows the expression of Brachury and (E) shows the loss of expression in the embryoid bodies after lipopolysaccharides exposure. Panel (F) shows the absence of HMGB1 induced by lipopolysaccharides. Panels (H) and (J) phase contrast pictures show control embryoid bodies with positive signs of mineralization detected by Alizarin Red and von Kossa staining, respectively. Panels (I) and (K) show phase contrast pictures indicating absence of mineralization in embryoid bodies pre-exposed to lipopolysaccharides, as detected by Alizarin Red and von Kossa staining, respectively. The pictures were acquired at 10× magnifications. Blue represents nuclei, green represents the antigens and their overlay gives cyan.

FIG. 7: Comet assay showing the degree of apoptosis induced in control and lipopolysaccharide-treated embryoid bodies. Panel (A) shows normal 5-day old embryoid bodies exposed to lipopolysaccharides for 48 hours. Pictures were acquired at 10× magnifications.

### DETAILED DESCRIPTION OF THE INVENTION

The present invention relates to an in vitro embryonic model comprising spherical smooth-embryoid body (SSE) for determining effect of molecule.

In another embodiment of the present invention, said model identifies stage of the SSE development at which the molecule acts.

In yet another embodiment of the present invention, said SSE is about 3-6 days old, preferably about 4.5 days old.

In still another embodiment of the present invention, said SSE is about 100-400 μm in diameter, preferably about 200-300 μM in diameter.

In still another embodiment of the present invention, said SSE is obtained from stem cell selected from a group comprising embryonic stem cells (ESCs), embryonic germ cells (EGCs) and embryonic carcinoma cells (ECCs), preferably human embryonic stem cells (hESCs).

In still another embodiment of the present invention said effect is selected from a group comprising embryotoxicity, development defects, lineage induction, formation of tissues, arrested growth, cell proliferation, epigenetic changes, chromosomal aberrations, karyotypic changes, cytotoxicity, cell migration, interaction with extracellular matrix components, effect on niche components of cells, mutagenesis, pharmacogenetic effects and toxicogenetic effects.

In still another embodiment of the present invention, said molecule is selected from a group comprising drugs, formulations, contraceptives, herbal extract or preparation, environment pollutants, endotoxins, nanoparticles, viruses, microbial toxins, biologicals, antibodies, proteins, DNA, RNA and siRNAs.

The present invention also relates to an in vitro method for determining effect of molecule on spherical smooth-embryoid body (SSE) comprising acts of:
a) exposing the molecule to the SSE, and
b)
   (i) screening for the effect of the exposure on formation and induction of germ lineages;
   (ii) screening for the effect of the exposure on germ lineages;
   (iii) screening for the effect of the exposure on implantating embryo;
   (iv) screening for the effect of the exposure on differentiation into tissue; and
(v) screening for cytotoxic effect of the exposure; or any combination(s) thereof.

In still another embodiment of the present invention, said method is carried out using the in vitro embryonic model.

In still another embodiment of the present invention said molecule is selected from a group comprising drugs, formulations, contraceptives, herbal extract or preparation, environment pollutants, endotoxins, biologicals, nanoparticles, viruses, microbial toxins, antibodies, proteins, DNA, RNA, and siRNAs.

In still another embodiment of the present invention said screening is carried out by studying expression of suitable markers.

In still another embodiment of the present invention said marker is selected from a group comprising lineage marker, pluripotency marker and epigenetic marker; or any combination(s) thereof.

In still another embodiment of the present invention said lineage marker is selected from a group comprising (a) ectoderm markers, (b) endoderm markers, (c) mesoderm markers, and (d) trophectoderm markers as given in table nos. 1, 2, 3 and 4 respectively:

In still another embodiment of the present invention said pluripotency marker is selected from a group comprising human embryonic stem cell specific signature and pluripotency genes as given in table 5.

In still another embodiment of the present invention said epigenetic marker is selected from a group comprising (a) imprinted genes and (b) candidate genes which can get methylated as given in table nos. 6 and 7 respectively.

In still another embodiment of the present invention, said expression of marker is studied by using techniques selected from a group comprising RT-PCR, flow cytometry and immunofluorescence.

The present invention also relates to an in vitro embryo implantation model comprising:
a) coat of extracellular matrix onto support matrix having well(s);
b) layer of endometrial cells onto the extracellular matrix; and
c) spherical smooth-embryoid body (SSE) placed into the well to determine effect of molecule.

In still another embodiment of the present invention, said model identifies stage of the SSE development at which the molecule acts.

In still another embodiment of the present invention, said extracellular matrix is selected from a group comprising fibronectin, collagen, matrigel, laminin, gelatin, albumin, poly-d-lysine, vitonectin and entactin.

In still another embodiment of the present invention, said support matrix is selected from a group comprising agar, low melting agarose, polyacrylamide, gelatin, collagen, chitosan and 3D collagen or polymer scaffolds, preferably agarose.

In still another embodiment of the present invention, said endometrial cell is selected from a group comprising mouse endometrial cell, human endometrial cell, rabbit endometrial cell, murine endometrial cell, porcine endometrial cell, bovine primary endometrial stromal cell and endometrial stromal cell lines, preferably mouse endometrial stromal cell and human endometrial stromal cell.

In still another embodiment of the present invention, said SSE is about 3-6 days old, preferably about 4.5 days old.

In still another embodiment of the present invention, said SSE is about 100-400 μm in diameter, preferably about 200-300 μm in diameter.

In still another embodiment of the present invention, said SSE is obtained from stem cell selected from a group comprising embryonic stem cells (ESCs), embryonic germ cells (EGCs), embryonic carcinoma cells (ECCs) preferably human embryonic stem cells (hESCs), preferably human embryonic stem cell (hESCs).

In still another embodiment of the present invention, said effect is selected from a group comprising embryotoxicity, detection of activities of drugs/biologicals which are (a) detrimental to embryonic development and pregnancy, (b) detrimental to lineage induction and tissue formation, (c) inhibit embryo implantation or attachment, (d) inhibit migration and invasion of cells, (e) beneficial for developing embryo, (f) improves attachment of the embryo, (g) improves lineage induction and tissue formation, (h) improves cell proliferation, (i) improves migration and invasion of cells and (j) modulates secretion of growth factors, cytokines and hormones, mutagenesis, pharmacogenetic effects and toxicogenetic effects.

In still another embodiment of the present invention, said molecule is selected from a group comprising drugs, formulations, contraceptives, herbal extract or preparation, environment pollutants, endotoxins, nanoparticles, viruses, microbial toxins, biologicals, antibodies, proteins, DNA, RNA and siRNAs.

The present invention also relates to an in vitro method of determining effect of lipopolysaccharide (LPS) on embryoid bodies (EBs), said method comprising acts of:
a. exposing the EBs to the LPS to trigger expression of gene HMGB1 in cytoplasm of the EBs, and
b. observing silencing of mesoderm induction and functional differentiation in the EBs.

In still another embodiment of the present invention, said silencing of mesoderm induction and functional differentiation leads to defect in formation of bone, blood and/or heart muscle.

In still another embodiment of the present invention, said expression of the gene HMGB 1 in nucleus of the EBs helps in maintenance of pluripotency in the EBs.

The present disclosure relates to the field of stem cells particularly development of a novel human embryonic model using human embryoid bodies obtained from the human embryonic stem cell. The novel human embryonic model disclosed thus can provide a screening assay for determining the toxic activity of the drugs. The assay is useful in identifying the stage of fetal development where the compound/drug can exert its detrimental effects.

As the embryo develops it goes through multiple stages of development and differentiation. The embryos differentiate into germ lineages, they implant on the maternal uterine endometrium and then the trophectoderm forms a placenta, and this is followed by further differentiation of the germ lineages to tissues. Applicant has developed an in-vitro embryo implantation model using human embryonic bodies obtained from human embryonic stem cells. The said model is equivalent for normal implantation mode and has been developed stage by stage using human EBs, extracellular matrix proteins like fibronectin, collagen and matrigel, on an agarose base. Thus this assay can help in identifying the stage of fetal development where the compound/drug can exert its detrimental effects.

The effect of the compound/drug was also tested in non-cavitating (early) and cavitating embryonic bodies (late) which are very similar to human embryo in term of its germ layer composition (ecto-, endo-, and mesoderm) and ability to give rise to different tissue type of the body.

The screening assay was carried out in five stages in correlation with increasing developmental complexities such as effect on the formation of germ lineages, effect on the germ lineages, effect on implantating embryos; effect on differentiation into tissues, cytotoxic effect.

The effects was monitored and evaluated by studying the expression of a set of developmentally important lineage markers given in tables 1, 2, 3, & 4. A set of epigenomic marker genes which are developmentally regulated, methylated and imprinted were identified (Table 6 and Table 7). A change in the expression pattern of any of these genes in the EBs or during the formation of the EBs as a result of an exposure to a drug indicates a possible developmental defect of the growing fetus. The cytotoxic and apoptotic effect of the drugs was assessed on hESCs using MTT [3-(4,5-dimethylthiazol-2-yl)-2,5-diphenyltetrazolium bromide] assay (Mosmann et al., 1983), DNA fragmentation in EBs through Comet assay, and Apoptosis was screened by expression analysis of genes like caspase-8, 3, p 73, p 53 etc.

This assay is useful to evaluate problem in normal differentiation into germ layer lineages, implantation failure, developmental/birth defects of specific tissue types, the overall embryotoxicity and cytotoxicity and apoptosis.

In developing this assay EBs were used as entities equivalent to a developing embryo to screen the effect of drugs/compounds on the developing embryos. This assay is useful to study the overall embryotoxic potential of a drug/molecule/compound/any formulation/herbal extract or preparation; specific effects of the drug/compound on any of the germ lineages leading to birth defects or possible abnormal growth and development of the fetus; the potential of the drug/compound to cause implantation failure or abortions in pregnant women; the effect of various diseases, infections, microbial toxins on the developing embryo; the effect of compounds in contraception research, drug development and screening; the effect of genital tract infections of any kind on the developing embryo; the cytotoxic potential of the drug; and the effect of environmental pollutants of the air, water or soil on fetal development.

### Effect on the Formation of Germ Lineages

Human Embryonic Stem Cells (hESCs) cultured on feeder cells of mouse or human origin, or cultured in feeder free conditions were exposed to several different dilutions of the drug for 6 days and subjected to Embryoid Body (EB) formation on non adherent plates, in presence of the drug for another 4 days. The control set was free from any extraneous addition of drugs. The formation of normal EBs in the control was evaluated by testing the expression of all the germ lineage, pluripotency and epigenomic markers as given in table Nos. 1-7 by RT-PCR, Flow Cytometry and Immunofluorescence. Alteration in the expression profiles of these genes in the treated EBs indicated a detrimental effect on embryonic development. A failure to form nicely cavitating EBs, as observed under the microscope, or a delay in the formation of the EBs as compared to the control indicated a possible embryo toxic effect of the drug.

The effect of molecules/compounds was screened. The compound, such as Rho Kinases inhibitor Y27632, 5-Azacytidine, and gram negative bacterial endotoxin lipopolysaccharide (LPS) on the formation of EBs from hESCs were screened for their toxic activity. LPS is known to be involved in genital tract infection related pathogenesis and pregnancy losses.

It was seen that hESCs exposed to 10-20 μM concentration of Y27632 delayed EB formation and the cell aggregates did not form cavities upto day 10 as observed under the microscope. Similarly, 5 μM concentration of 5-Azacytidine, and LPS at a concentration of 10 pg/ml and above showed complete inhibition of EB formation from the hESCs. Based on these results it was concluded that these three molecules have a dose dependent effect on the differentiation of the hESCs to the germ lineages.

### Effect on the Germ Lineages:

Day 4 to 10 old EBs were maintained for 2 to 10 days in presence or absence of various (atleast 8 different) dilutions of the drug supplemented to the standard EB culture media. At the end of the incubation period the EBs were collected and the RNA is isolated from both the control and treated groups. The RNA was used for RT-PCR analysis of a set of developmentally important genes, pluripotency markers like (Nanog, Oct4, Sox2, and HMGB 1), lineage markers for ectoderm, endoderm, mesoderm and trophectoderm listed in table 1, 2, 3, 4, and also for a set of epigenetic signature genes (table nos. 6-7) identified by applicant. Alteration in the expression patterns of these genes in presence or absence of the compound in these EBs indicates their embryotoxic effect and potential to perturb the formation of specific or multiple lineages.

Four day old EBs were exposed to gram negative bacterial endotoxins/LPS at a concentration of 12 pg/ml for 2 to 4 days. The EBs were collected and analysed for pluripotency markers like Oct4, Sox2, HMGB1, and lineage markers like Nestin, PIII-tubulin, GATA4, BMP2, Brachury, Hand1 and BMP4 were studied, by RT-PCR. Positive expression for ecto-, endo-, and mesoderm lineages markers Nestin, 13111-tubulin, GATA4, BMP2, BMP4, Hand 1 and Brachury were found in all the normal EBs. HMGB 1 expression was not found in these normal EBs. The LPS treated EBs also showed the positive expression of Nestin, βIII-tubulin, and GAT A4. However, the mesoderm markers BMP2, Hand1, BMP4 and Brachury were silenced in the endotoxin treated EBs. The treated EBs also showed a positive expression of HMGB 1. The silencing of the mesoderm specific genes like Brachury, BMP2, BMP4 and Hand1 in the EBs after treatment with endotoxin indicates that the presence of LPS in the environment of the developing embryo can lead to defect in the formation of a functional mesoderm. This also explains many birth defects, which occurs as a result of gram negative bacterial infections. The expression of the LPS inducible cytokine and pluripotency associated gene HMGB1 in the EBs upon LPS exposure indicate its probable role in the formation of poorly formed embryos during such infections.

### Effect on Implantating Embryos:

An in vitro 3-dimensional (3-D) model/system for embryo implantation employing EBs in place of embryos was developed. The EBs collected on different days were subjected to implantation/attachment on the artificial substratum/surface.

To make this artificial surface regular tissue culture dishes or organ culture dishes were coated with about 1 to 2 mm thick coating of 0.5 to 1% low melting or high melting agarose in DPBS. A few wells of about 1 to 2 mm diameter are created using sterile paper disks, preferable in the center of the dish, which were removed after the agarose solidifies. Alternatively a horizontal tube like structure can be created by casting the gel over a fine glass capillary, which can be withdrawn as the agarose solidifies. This cavity/depression or tube like structure of agarose is then coated with matrix proteins like matrigel, collagen, fibronectin, laminin, gelatin etc. The coated agarose dishes were also layered with mouse or human endometrial stromal cells obtained from primary cultures or cell lines. The EBs were placed carefully in the cavity created or flushed inside the tube using a bent pasture pipette. The cells were cultured in normal standard EB media.

The EBs adhere and outgrows spreading out as a monolayer of cells on the extracellular matrix. Some cells invaded the surrounding agarose by day 10. A failure to implant/outgrow within ten days on this matrix after exposure to the drug/molecule indicates an abnormality. However, some drugs/could also support/enhance the process of attachment and outgrowth of the EBs. These molecules were screened for several other possible applications, though these may not necessarily be safe for embryonic development.

For an unbiased evaluation of the effect of all kinds of drugs/compounds, expression of a set of molecular markers for lineages, pluripotency and epigenetically regulated and or imprinted genes listed in tables 1 to 6 were screened. The molecular signature or gene expression profile for normal EBs after outgrowth at day 10 were determined by RT-PCR followed by a comparison with the EBs cultured in presence of various dilutions of the drug for ten days.

Four day old EBs were cultured on agarose and fibronectin coated surfaces. The control group showed attachment and extensive spreading of cells on the surface from the 4^{th} day onwards. The other group treated with 20 μM concentration of the ROCK inhibitor drug Y27632, showed an enhanced attachment from day 2 onwards. However, gram negative bacterial LPS completely inhibited such an attachment and outgrowth at a concentration of 15 pg/ml and above. This indicates that LPS has a detrimental effect on implantation. This has been proved in several earlier studies in an in vivo mouse model (Deb et al., 2004, 2005, 2006, 2007). However, as Y27632 supports and enhances outgrowth, the effect of this drug on the expression profiles of the lineage markers and other genes needs to be screened for evaluating effect on normal development.

Effect on Differentiation into Tissues:

The EBs were used to direct their differentiation into tissues of the ectoderm (nerve, skin), endoderm (pancreas, lungs), mesoderm (bone, blood, cardiomyocytes) and trophectoderm lineages (placenta) using known and published methods (Bader et al., 2000; Buttery et al., 2001; Lumelsky et al., 2001; Lee et al; 2000; Schuliner et al., 2001). A description of the methods used for differentiation of hESC to various tissues of the ectoderm, endoderm, mesoderm and trophectoderm lineages can be found in Hyslop et al. (2005). The ability of the compound/drug to inhibit the differentiation of EBs into these tissues of any particular type will indicate the possibility of developmental defect induced by the drug.

Endotoxins/LPS silenced the expression of mesoderm specific genes in the EBs. This indicates that the LPS exposed EBs are defective of a functional mesoderm. To confirm this effect the LPS exposed EBs were directed towards tissues of mesoderm origin like cardiomyocytes, blood or bone. A failure to differentiate into anyone of the tissue types of mesoderm origin confirmed the fact. This indicates the possibility of defects in the blood, bone or heart formation as a result of endotoxin exposure during fetal development.

### Cytotoxic Effect:

The cytotoxicity of the drugs was evaluated by MMT assay in the hESCs. DNA fragmentation in EBs was evaluated by comet assay (Deb et al., 2007), followed by analysis of expression of apoptotic genes like Caspases-8 and 3, p 53, p 73 by RTPCR. In this study we have used embryoid bodies as an in vitro model to examine the effect of LPS on the differentiation and faithful induction of the germ lineages during peri-implantation embryonic development. The expression of LPS inducible and pluripotency related gene high mobility group box 1 (HMGB1) was studied to assess its possible involvement in the aberrant differentiation of the LPS treated EBs [12, 13]. HMGB1 is explicitly expressed by the cells of the inner cell mass and is absent in the trophectoderm cells of the blastocyst [14]. HMGB1 is also known as a DNA-binding protein which can regulate expression of genes [12]. Because of its versatile roles both during development and in response to endotoxins, we hypothesized that HMGB1 may be a key player in mediating LPS induced developmental defects. We found that LPS exposure for 48 hrs inhibited functional mesoderm formation in these EBs. LPS induced HMGB1 expression in these EBs also indicates its possible role in silencing mesoderm induction. These findings for the first time indicate that the presence of endotoxins in the maternal environment can lead to predictable mesoderm tissue-specific birth defects like malformation of bones. This study also indicates that HMGB1 is related to pluripotency in hESCs and that its expression silences mesoderm specific genes and differentiation.

EBs derived from the human embryonic stem cell (hESC) line HUES9 were exposed to 12.5 pg/ml of LPS for 48 hrs. The expression profile of the ectoderm, endoderm, mesoderm and trophectoderm lineage markers like βIII-tubulin, GATA4, BMP2, Brachury and β-hCG were studied, by RT-PCR and Immunofluorescence. Inhibition of mesoderm induction was confirmed by RT-PCR analysis for hANP, cTnT, ABCG2, GATA2, BMP4 and HAND1. Osteoblast differentiation was induced in the EBs, and confirmed by von Kosa and Alizarin red staining. A comet assay was also done to assess the degree of apoptosis in these EBs.

It was found that the LPS treated EBs were selectively silenced for mesoderm markers and failed to differentiate into functional osteoblasts. HMGB1 expression was absent in the normal EBs and was found to be localized in the cytoplasm of the LPS-treated EBs. Overall, our data indicates that endotoxin-induced HMGB1 expression in the peri-implantation stage embryos can bring about severe birth defects of the bone, heart etc. This study also indicates that HMGB1 could be involved in maintenance of pluripotency in the hESCs by impeding their differentiation.

Genital tract infection is a predominant cause for birth anomalies both in cases of normal conception or after assisted reproductive techniques (ART) [19]. Several of these infections are caused by gram-negative bacteria like Chlamydia trachomatis which are asymptomatic and cause chronic upper tract infections [20]. In seventy percent of the birth defect cases the underlying causes are unknown. Here we have studied the effect of Gram-negative bacterial vaginosis on aberrant fetal development using an embryonic stem cell model. During such injections the preimplantation embryos are exposed to bacterial endotoxins/LPS in the environment [11, 8]. The effect of LPS on preimplantation embryonic development and subsequent failure of implantation has been widely studied in animal and rodent models [10, 11]. Studies on the underpinning mechanisms leading to developmental abnormalities in human embryos are not possible due to ethical limitations of the use of human embryos.

First, characterization of the day 5 old EBs for the presence of all the germ layer lineages was done. The positive expression of βIII-tubulin, GATA4, BMP2, Brachury and β-hCG indicated the presence of all the germ layer lineages (the endo-, ecto-, meso-, and trophectoderm) in these EBs. This also established the fact that these EBs were equivalent to developing peri-implantation stage embryos in term of their constituent cells representing all the lineages. These similarities between developing embryos and EBs derived from hESCs have been established by previous workers [4]. In the present study LPS was supplemented in the culture media at a concentration of 12.5 pg/ml. This dose was arbitrarily chosen and is more than twice of a report by [21] which showed that as low as 2-5 pg/ml of LPS was enough to cause alterations in the proliferation of hematopoietic precursor cells in culture [21].

The effect of LPS on the induction of the germ lineages in the EBs were studied by RT-PCR and immunoflurescence analyses of the lineage specific markers. We found a specific silencing of all the eight mesoderm markers namely Brachury, BMP2, hANP, cTnT, ABCG2, GATA2, BMP4 and HAND1. The mesoderm lineage is known as precursors for tissues like the osteogenic cells, haematopoietic precursor cells, and [22]. Defects in bone and muscles are very common birth defects and their underlying causes largely remain unknown. This study for the first time provides an in vitro human model for such studies and indicates towards a role of LPS for such abnormalities.

In this study it was found that HMGB1 was not expressed in the normal EBs and its expression was induced in the EBs treated with LPS. HMGB1 is a known LPS inducible cytokine [12], and its cytoplasmic expression in the LPS treated EBs indicate its possible role as a non-classical proinflammatory cytokine, in causing the mesodermal defects. Anti-HMGB1 antibodies can be used to treat lethal endotoxemia and sepsis [12]. Whether this intervention could be effective for protecting the developing fetus from the adverse effects of endotoxins is not known. At the same time the observed nuclear localization and expression of HMGB1 in the pluripotent hESCs and its loss of expression in a differentiated population of cells in the EBs, indicate towards its probable involvement in maintenance of sternness in the hESCs. This observation is in support of a previous study which showed that HMGB1 is specifically expressed in the inner cell mass of the blastocyst [14]. Our data also indicates that nuclear or DNA binding form of HMGB1 may be instrumental in silencing differentiation to the various lineages and thus maintains pluripotency in the hESC lines. Further studies on establishing HMGB1 as a pluripotency marker is currently underway in our laboratory.

It was found that the average number of cells per EB in both the LPS treated and the control EBs were not significantly different. This indicates that the dose of LPS used in this study did not interrupt the cell divisions or the process of formation of the EBs. The specific silencing of mesodermal genes therefore possibly indicates a reprogramming of genes involved in the differentiation and induction of germ lineages during development. The comet assay showed more DNA tailing or fragmentation in the LPS treated EBs as compared to the control. This indicates that many of the cells in the EBs were already undergoing apoptosis as an effect of LPS. We also noticed that during the induction of osteogenic differentiation in the control and treated EBs, no differences were found in their efficiency for attachment and proliferation. However, the LPS treated EBs failed to undergo osteoblast differentiation as confirmed by the absence of mineral deposition staining like von Kossa and Alizarin Red. It is however not clear if the LPS induced apoptosis in the EBs was exclusively selective towards the population of cells which were of mesoderm origin. The molecular mechanism for the selective mesoderm silencing and the possible role of HMGB1 needs to be deciphered.

The present study for the first time demonstrates a correlation between gram-negative bacterial LPS and birth defects related to formation of tissues of mesoderm origin like the bones, blood and or heart-muscles. We also show that early EBs could be effectively employed as a model system to study fetal abnormalities caused due to maternal infections or due to new drugs. Expression and cytoplasmic localization of the DNA-binding cytokine HMGB1 in the EBs after LPS exposure indicates towards its probable involvement in the formation of developmentally compromised embryos during such infections. Our finding at the same time strongly indicates that nuclear localization of HMGB1 maintains pluripotency in hESCs by inhibiting the faithful induction of all the germ layer lineages.

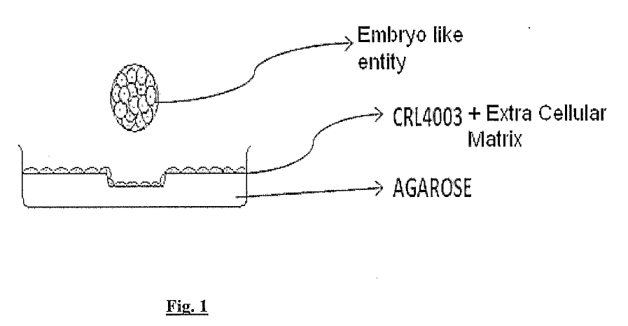

The model consists of a culture plate which is coated with Low melting (0.5%) agarose, A well of various depths is created in the agarose by various means eg: putting sterile filter paper disks while casting the gel and removing them latter. The gel is then coated with extracellular matrix components like fibronectin, collagen, laminin, etc. Human endometrial stromal fibroblast cell line CRL4003 is grown on this extracellular matrix as a monolayer. The spherical embryoid bodies or embryo like entities at day 4.5 are injected or pipetted into these cavities. The attachment is allowed in a culture media regularly used for hESC culture, without FGF2 supplementation (FIG. 1).

The depth and diameter of the well created can vary and will depend on the type of test to be addressed.
A deeper well (1 to 2 mm deep) is required to test the ability of the cells to invade into the agarose ie., to carry out the invasion assays.
A relatively shallow well (lesser than 1 mm) is required for the attachment assay.

The choice of the extracellular matrix coating will also depend of the specific question being asked. We use fibronection, laminin, collagen or a combination of all (in required ratios).

### Example

Fibronectin is most preferred while testing the ability of the trophectoderm cells in the EBs to invade or attach into the maternal stromal cells.

Various applications of the In vitro model and its design are as follows:

1. Effect of compounds on rate of attachment and outgrowth of the spherical cytic/cavitating or non-cavitating EBs can be studied using our in vitro implantation model
Negative effect will show inhibition of attachment or slower rate of outgrowth and hence detrimental to pregnancy outcome.
Positive effect will show enhanced attachment and faster rate of outgrowth and hence supportive to pregnancy outcome.

2. Cell Invation/migration assays: Whether a compound or biologics can enhance the invasion/migration of fetal cells into the maternal stromal environment can be studied using this model. In other words this model can be used as a tool to study the fetal maternal interactions. The extent of invasion of the cells through the agarose gel can be observed by staining the cells with DAPI/Hoechst or any other live cell tracing dye, and the areas over time can be calculated using a fluorescence microscope.

3. Lineage induction studies: The embryoid bodies after attachment to the implantation site differentiates to all lineages. In presence of a drug/compound/biologicals this ability to give rise to all lineages may be compromised (detrimental effect, e.g. LPS, DMSO, H2O2 etc) or remain unaltered (safe compound, Eg: Gold, silver nanoparticles)

TABLE 1
A list of ectoderm markers

| Sl. No | Gene Symbol | Gene Description | NCBI-Gene ID |
|---|---|---|---|
| 1. | PAX6 | Paired Box Gene 6(Aniridia, Keratitis) | 5080 |
| 2. | VIM | Vimentin | 7431 |
| 3. | CRABP2 | Cellular Retinoic Acid Binding Protein 2 | 1382 |
| 4. | SEMA3A | Sema Domain, Immunoglobulin Domain (Ig), | 10371 |
| | | Short Basic Domain, Secreted, (Semaphorin) 3A | |
| 5. | MSI1 | Musashi Homolog 1 (Drosophila) | 4440 |
| 6. | MAP2 | Microtubule-Associated Protein 2 | 4133 |
| 7. | GFAP | Glial Fibrillary Acidic Protein | 2670 |
| 8. | OLIG2 | Oligodendrocyte Lineage Transcription Factor 2 | 10215 |
| 9. | NES | Nestin | 10763 |
| 10. | NEUROD1 | Neurogenic Differentiation 1 | 4760 |
| 11. | TH | Tyrosine Hydroxylase | 7054 |
| 12. | TUBB3 | Tubulin, Beta 3 | 10381 |

TABLE 2
A list of endoderm markers

| Sl. No | Gene Symbol | Gene Description | NCBI-Gene ID |
|---|---|---|---|
| 1. | ACVR1B | Activin A Receptor, Type Ib | 91 |
| 2. | AFP | Alpha-Fetoprotein | 174 |
| 3. | DCN | Decorin | 1634 |
| 4. | FABP2 | Fatty Acid Binding Protein 2, Intestinal | 2169 |
| 5. | FGF8 | Fibroblast Growth Factor 8 (Androgen-Induced) | 2253 |
| 6. | FLT1 | Fms-Related Tyrosine Kinase 1 | 2321 |
| 7. | FN1 | Fibronectin 1 | 2335 |
| 8. | FOXA2 | Forkhead Box A2 | 3170 |
| 9. | GATA4 | Gata Binding Protein 4 | 2626 |
| 10. | GATA6 | Gata Binding Protein 6 | 2627 |
| 11. | GCG | Glucagon | 2641 |
| 12. | H19 | H19, Imprinted Maternally Expressed | 283120 |
| | | Untranslated mRNA | |
| 13. | HGF | Hepatocyte Growth Factor (Hepapoietin A; | 3082 |
| | | Scatter Factor) | |
| 14. | HNF4A | Hepatocyte Nuclear Factor 4, Alpha | 3172 |
| 15. | INS | Insulin | 3630 |
| 16. | LAMB1 | Laminin, Beta 1 | 3912 |
| 17. | LAMC1 | Laminin, Gamma 1 (Formerly Lamb2) | 3915 |
| 18. | PECAM1 | Platelet/Endothelial Cell Adhesion Molecule | 5175 |
| | | (Cd31 Antigen) | |
| 19. | SERPINA1 | Serpin Peptidase Inhibitor, Clade A (Alpha-1 | 5265 |
| | | Antiproteinase, Antitrypsin), Member 1 | |

TABLE 3
A list of mesoderm markers.

| Sl. No | Gene Symbol | Gene Description | NCBI-Gene ID |
|---|---|---|---|
| 1. | BRACHYURY | T, Brachyury Homolog (Mouse) | 6862 |
| 2. | COL1A1 | Collagen, Type I, Alpha I | 1277 |
| 3. | HAND1 | Heart And Neural Crest Derivatives Expressed 1 | 9421 |
| 4. | COL2A1 | Collagen, Type II, Alpha 1 (Primary | 1280 |
| | | Osteoarthritis, Spondyloepiphyseal Dysplasia, | |
| | | Congenital) | |
| 5. | HBZ | Hemoglobin, Zeta | 3050 |
| 6. | WT1 | Wilms Tumor 1 | 7490 |
| 7. | MYF5 | Myogenic Factor 5 | 4617 |
| 8. | DES | Desmin | 1674 |
| 9. | NPPA | Natriuretic Peptide Precursor A | 4878 |
| 10. | HBB | Hemoglobin, Beta | 3043 |
| 11. | RUNX2 | Runt-Related Transcription Factor 2 | 860 |
| 12. | BMP2 | Bone Morphogenetic Protein 2 | 650 |
| 13. | IGF2 | Insulin-Like Growth Factor 2 (Somatomedin A) | 3481 |

TABLE 4
A list of trophectoderm markers

| Sl. | Gene | | NCBI- |
|---|---|---|---|
| No | Symbol | Gene Description | Gene ID |
| 1. | CDX2 | Caudal Type Homeobox Transcription | 1045 |
| | | Factor 2 | |
| 2. | GATA2 | GATA Binding Protein 2 | 2624 |
| 3. | hCG-beta | Chorionic Gonadotropin, Beta Polypeptide | 1082 |
| 4. | EOMES | Eomesodermin Homolog (Xenopus Laevis) | 8320 |
| 5. | GCM1 | Glial Cells Missing Homolog 1 | 8521 |
| | | (Drosophila) | |
| 6. | KRT1 | Keratin 1 (Epidermolytic Hyperkeratosis) | 3848 |
| 7. | TBX1 | T Box 1 | 6811 |
| 8. | PSG3 | Pregnancy Specific Beta-1-Glycoprotein 3 | 5671 |
| 9. | HAND1 | Heart And Neural Crest Derivatives | 9421 |
| | | Expressed 1 | |
| 10. | KRT18 | Keratin 18 | 3875 |
| 11. | EOMES | Eomesodermin Homolog (Xenopus Laevis) | 8320 |

TABLE 5
Human embryonic stem cell-specific signature and pluripotency genes

| Sl. No. | Gene Symbol | Gene Description | Gene ID |
|---|---|---|---|
| 1. | BMPR1A | Bone Morphogenetic Protein Receptor, Type IA | 657 |
| 2. | BRIX | Brix Domain Containing 2 | Hs.38114 |
| 3. | BYSL | Bystin-Like | 705 |
| 4. | CCNB1 | Cyclin B1 | 891 |
| 5. | CCND1 | Cyclin D1 | 595 |
| 6. | CCNE1 | Cyclin E1 | 898 |
| 7. | CD24 | CD24 Antigen (Small Cell Lung Carcinoma | 934 |
| | | Cluster 4 Antigen) | |
| 8. | CD9 | CD9 Antigen (P24) | 928 |
| 9. | CDC2 | Cell Division Cycle 2, G1 To S And G2 To M | 983 |
| 10. | CDH1 | Cadherin 1, Type 1, E-Cadherin (Epithelial) | Hs.461086 |
| 11. | CDK4 | Cyclin-Dependent Kinase 4 | 1019 |
| 12. | CHK2 | CHK2 Checkpoint Homolog | 11200 |
| 13. | CHST4 | Carbohydrate (N-Acetylglucosamine 6-O) | Hs.251383 |
| | | Sulfotransferase | |
| 14. | CKMT1A | Creatine Kinase, Mitochondrial (Ubiquitous) | 548596 |
| 15. | CKMT1B | Creatine Kinase, Mitochondrial (Ubiquitous) | 1159 |
| 16. | CLDN6 | Claudin 6 | Hs.533779 |
| 17. | COMMD3 | COMM Domain Containing | Hs.534398 |
| 18. | CRABP1 | Cellular Retinoic Acid Binding Protein 1 | 1381 |
| 19. | CX43 | Gap Junction Protein, Alpha 1, 43 kda (Connexin | 2697 |
| | | 43) | |
| 20. | CX45 | Gap Junction Protein, Alpha 7, 45 kda (Connexin | 10052 |
| | | 45) | |
| 21. | DIAPH2 | Diaphanous Homolog (Drosophila) | Hs.226483 |
| 22. | DNMT3B | DNA (Cytosine-5-)-Methyltransferase 3- | Hs.251673 |
| 23. | DPPA5 | Developmental Pluripotencyassociated | Hs.125331 |
| 24. | EDNRB | Endothelin Receptor Type | Hs.82002 |
| 25. | EPHA1 | EPH Receptor A1 | 2041 |
| 26. | EPHA2 | EPH Receptor A2 | 1969 |
| 27. | EPHA4 | EPH Receptor A4 | 2043 |
| 28. | EPHB4 | EPH Receptor B4 | 2050 |
| 29. | ESG1 | Transducin-Like Enhancer Of Split 1 (E(Sp1) | 7088 |
| | | Homolog, Drosophila) | |
| 30. | FGF13 | Fibroblast Growth Factor 13 | 2258 |
| 31. | FGF2 | Fibroblast Growth Factor 2 (Basic) | 2247 |
| 32. | FGF4 | Fibroblast Growth Factor (Heparin Secretory | Hs.1755 |
| | | Transformingprotein 1, Kaposi Sarcoma | |
| | | Oncogene) | |
| 33. | FGFR1 | Fibroblast Growth Factor Receptor 1 (Fms-Related | 2260 |
| | | Tyrosine Kinase | |
| 34. | FGFR2 | Fibroblast Growth Factor Receptor 2 (Bacteria- | 2263 |
| | | Expressed Kinase, Keratinocyte Growth Factor | |
| | | Receptor, Craniofacial Dysostosis | |
| 35. | FGFR4 | Fibroblast Growth Factor Receptor 4 | 2264 |
| 36. | FOXD3 | Forkhead Box D3 | Hs.546573 |
| 37. | FST | Follistatin | 10468 |
| 38. | FZD5 | Frizzled Homolog 5 (Drosophila) | 7855 |
| 39. | FZD7 | Frizzled Homolog 7 (Drosophila) | 8324 |
| 40. | GABABR1 | Gamma-Aminobutyric Acid (GABA) B Receptor, 1 | 2550 |
| 41. | GABRB3 | Gamma-Aminobutyric Acid (GABA) A Receptor, | 2562 |
| | | Beta 3 | |
| 42. | GAL | Galanin | Hs.278959 |
| 43. | GBX2 | Gastrulation Brain Homeo Box | Hs.184945 |
| 44. | GDF3 | Growth Differentiation Factor | Hs.86232 |
| 45. | GJA1 | Gap Junction Protein, □-1, 43 Kda (Connexin | Hs.74471 |
| | | 43) | |
| 46. | GPC4 | Glypican 4 | 2239 |
| 47. | GRB7 | Growth Factor Receptor-Bound Protein | Hs.86859 |
| 48. | GYLTL1B | Glycosyltransferase-Like 1B | Hs.86543 |
| 49. | HMGB1 | High-Mobility Group Box 1 | 3146 |
| 50. | HOXA11 | Homeobox A11 | 3207 |
| 51. | IFITM1 | Interferon Induced Transmembrane Protein (9-27) | Hs.458414 |
| 52. | IFITM2 | Interferon Induced Transmembrane Protein (1-8D) | Hs.174195 |
| 53. | IMP- | IGF-II Mrna-Binding Protein | Hs.35354 |
| 54. | ITGA6 | Integrin, Alpha 6 | 3655 |
| 55. | ITGB1 | Integrin, □-(Fibronectin Receptor, -Polypeptide, | Hs.429052 |
| | | Antigen CD29 Includes MDF2, MSK12) | |
| 56. | ITGB1BP3 | Integrin &#2; -Binding Protein | Hs.135458 |
| 57. | JMJ | Jumonji, AT Rich Interactive Domain 2 | 3720 |
| 58. | KIT | V-Kit Hardy-Zuckerman Feline Sarcoma Viral | Hs.479754 |
| | | Oncogene | |
| 59. | KLF2 | Kruppel-Like Factor 2 | 10365 |
| 60. | KLF3 | Kruppel-Like Factor 3 | 51274 |
| 61. | KLF5 | Kruppel-Like Factor 5 | 688 |
| 62. | KLF9 | Kruppel-Like Factor 9 | 687 |
| 63. | LAMR1 | Ribosomal Protein SA | 3921 |
| 64. | LCK | Lymphocyte-Specific Protein Tyrosine Kinase | Hs.470627 |
| 65. | LECT1 | Leukocyte Cell-Derived Chemotaxin | Hs.421391 |
| 66. | LEFTY1 | Left-Right Determination, Factor | Hs.278239 |
| 67. | LEFTY2 | Left-Right Determination Factor (LEFTY2) | Hs.520187 |
| 68. | LIN28 | Lin-28 Homolog (Caenorhabditis Elegans) | Hs.86154 |
| 69. | MCM3 | MCM3 Minichromosome Maintenance Deficient 3 | 4172 |
| 70. | MSH2 | Muts Homolog 2, Colon Cancer, Nonpolyposis | 4436 |
| | | Type 1 | |
| 71. | NANOG | Nanog Homeobox | 79923 |
| 72. | NODAL | Nodal Homolog (Mouse) | Hs.370414 |
| 73. | NOG | Noggin | Hs.248201 |
| 74. | NR5A2 | Nuclear Receptor Subfamily 5, Group A, Member | Hs.33446 |
| 75. | NR6A1 | Nuclear Receptor Subfamily 6, Group A, | Hs.20131 |
| | | Member | |
| 76. | NTS | Neurotensin | Hs.80962 |
| 77. | NUMB | Numb Homolog (Drosophila) | Hs.509909 |
| 78. | PATCHED2 | Patched Homolog 1 (Drosophila) | 5727 |
| 79. | PEA3 | Ets Variant Gene 4 (E1A Enhancer Binding | 2118 |
| | | Protein, E1AF) | |
| 80. | PECAM | Platelet/Endothelial Cell Adhesion Molecule | 5175 |
| | | (CD31 Antigen) | |
| 81. | PITX2 | Paired-Like Homeodomain Transcription Factor 2 | 5308 |
| 82. | PMAIP1 | Phorbol-12-Myristate-13-Acetate-Induced | 5366 |
| | | Protein | |
| 83. | PODXL | Podocalyxin-Like | Hs.16426 |
| 84. | OCT4/ | POU Domain, Class 5, Transcription Factor | Hs.249184 |
| | POU5F1 | | |
| 85. | PSIP1 | PC4 And SFRS1 Interacting Protein 1 | 11168 |
| 86. | PTEN | Phosphatase And Tensin Homolog(Mutated In | Hs.500466 |
| | | Multiple Advanced Cancers 1) | |
| 87. | REST | RE1-Silencing Transcription Factor | Hs.401145 |
| 88. | REX1 | REX1, RNA Exonuclease 1 Homolog (S. Cerevisiae) | 57455 |
| 89. | SALL1 | Sal-Like 1 (Drosophila) | 6299 |
| 90. | SALL2 | Sal-Like 2 (Drosophila) | 6297 |
| 91. | SCGB3A2 | Secretoglobin, Family 3A, Member | s.483765 |
| 92. | SFRP2 | Secreted Frizzled-Related Protein | Hs.481022 |
| 93. | SMAD2 | SMAD Family Member 2 | Hs.12253, |
| 94. | SOX2 | SRY (Sex Determining Region Y)- | Hs.518438 |
| 95. | TDGF1 | Teratocarcinoma-Derived Growth Factor | Hs.385870 |
| 96. | TERF1 | Telomeric Repeat Binding Factor (NIMA- | 7013 |
| | | Interacting) 1 | |
| 97. | TERT | Telomerase Reverse Transcriptase | Hs.492203 |
| 98. | TFCP2L1 | Transcription Factor CP2-Like | Hs.156471 |
| 99. | TGFB1 | Transforming Growth Factor, Beta 1 (Camurati- | 7040 |
| | | Engelmann Disease | |
| 100. | TOP2A | Topoisomerase (DNA) II Alpha 170 kda | 7153 |
| 101. | TTF1 | Transcription Termination Factor, RNA | 7270 |
| | | Polymerase I | |
| 102. | UNG | Uracil-DNA Glycosylase | 7374 |
| 103. | UTF1 | Undifferentiated Embryonic Cell Transcription | Hs.458406 |
| | | Factor | |
| 104. | WNT1 | Wingless-Type MMTV Integration Site Family, | 7471 |
| | | Member 1 | |
| 105. | WNT5A | Wingless-Type MMTV Integration Site Family, | 7474 |
| | | Member 5A | |
| 106. | ZFP42 | Zinc Finger Protein 42 | Hs.335787 |
| 107. | ZNF206 | Zinc Finger Protein 206 | Hs.334515 |
| 108. | ZNF43 | Zinc Finger Protein 43 | 7594 |

TABLE 6
A list of Imprinted genes

| Sl. No. | Gene Symbol | Gene Description | NCBI-Gene ID |
|---|---|---|---|
| 1. | GRB10 | Growth Factor Receptor-Bound Protein 10 | 2887 |
| 2. | MEG3 | Maternally Expressed 3 | 55384 |
| 3. | MEST | Alpha/Beta Hydrolase Fold Family, Mesoderm | 4232 |
| | | Specific Transcript Homolog (Mouse) | |
| 4. | TP73 | Tumor Related Protein, Tumor Protein P73 | 7161 |
| 5. | DLK1 | Delta-Like 1 Homolog | 8788 |
| 6. | XIST | X (Inactive)-Specific Transcript | 7503 |
| 7. | ASB4 | Ankyrin Repeat And SOCS Box | 51666 |
| 8. | ASCL2 | Achaete-Scute Complex Homolog 2 (Drosophila) | 430 |
| 9. | ATP10A | Atpase, Class V | 57194 |
| 10. | CALCR | Calcitonin Receptor | 799 |
| 11. | CD81 | Transmembrane 4 Superfamily | 975 |
| 12. | CDKN1C | Cyclin-Dependent Kinase Inhibitor | 1028 |
| 13. | COMMD1 | Copper Metabolism Gene Murr 1 | 150684 |
| 14. | COPG2 | Coatomer Protein Complex, Submit Gamma 2 | 26958 |
| 15. | COPG21T1 | Coatomer Protein Complex, Subunit Gamma 2, | 53844 |
| | | Intronic Transcript 1 | |
| 16. | CPA4 | Carboxypeptidase | 51200 |
| 17. | CTNNA3 | PD NR M Catenin, Alpha 3 catenin (Cadherin- | 29119 |
| | | Associated Protein), Alpha 3 | |
| 18. | DCN | Proteoglycan, Decorin | 1634 |
| 19. | DIO3 | Deiodinase, Iodothyronine Type III | 1735 |
| 20. | DLX5 | Distal-Less Homeobox 5, Homeo Box- | 1749 |
| | | Containing | |
| 21. | GABRA5 | Gamma-Aminobutyric Acid Receptor | 2558 |
| 22. | GABRB3 | Gamma-Aminobutyric Acid Receptor | 2562 |
| 23. | GABRG3 | Gamma-Aminobutyric Acid Receptor | 2567 |
| 24. | GATM | Glycine Amidinotransferase | 2628 |
| 25. | GNAS | Neuroendocrine Secretory Protein 55 | 2778 |
| 26. | H19 | H19, Imprinted Maternally Expressed | 283120 |
| | | Untranslated Mrna | |
| 27. | HTR2A | Serotonin Receptor, 5-Hyroxytryptamine | 3356 |
| | | (Serotonin) Receptor 2A | |
| 28. | HYMAI | Hydatidiform Mole Associated and Imprinted | 57061 |
| 29. | IGF2 | Insulin-Like Growth Factor 2, Insulin-Like | 3481 |
| | | Growth Factor 2 (Somatomedin A) | |
| 30. | IGF2AS | Insulin-Like Growth Factor 2 Antisense | 51214 |
| 31. | IGF2R | Insulin-Like Growth Factor Receptor 2 | 3482 |
| 32. | IMPACT | Imprinted And Ancient | 55364 |
| 33. | INPP5F | V2 Isoform Only Inositol Phosphatase, Inositol | 22876 |
| | | Polyphosphate-5-Phosphatase F | |
| 34. | INS | Insulin | 3630 |
| 35. | KCNQ1 | KCNQ1 Overlapping Transcript 1 | 10984 |
| 36. | KCNQ1OT1 | Voltage-Gate Potassium Channel, Potassium | 3784 |
| | | Voltage-Gated Channel, KQT-Like Subfamily, | |
| | | Member 1 | |
| 37. | L3MBTL | Polycomb Group, L(3)Mbt-Like (Drosophila) | 26013 |
| 38. | LOC388015 | Retrotransposon-Like 1 | 388015 |
| 39. | MAGEL2 | MAGE-Like Protein, MAGE-Like 2 | 54551 |
| 40. | MKRN3?″ | Makorin, Ring Finger Protein | 7681 |
| 41. | NAP1L4 | Nucleosome Assembly Protein, Nucleosome | 4616 |
| | | Assembly Protein 1-Like 4 | |
| 42. | NAP1L5 | Nucleosome Assembly Protein, Nucleosome | 266812 |
| | | Assembly Protein 1-Like 5 | |
| 43. | NDN | Needin, Neuronal Growth Suppressor, Needin | 4672 |
| | | Homolog (Mouse) | |
| 44. | NNAT | Neuronatin | 4826 |
| 45. | OSBPL5 | Oxysterol Binding Protein-Like 5 | 114879 |
| 46. | PEG10 | Retroviral Gag Pol Homologue, Paternally | 23089 |
| | | Expressed 10 | |
| 47. | PEG3 | Zinc-Finger Protein, Paternally Expressed 3 | 5178 |
| 48. | PHEMX | Tetraspanin Superfamily, Tetraspanin 32 | 10077 |
| 49. | PHLDA2 | Pleckstrin Homology-Like Domain, Pleckstrin | 7262 |
| | | Homology-Like Domain, Family A, Member 2 | |
| 50. | PLAGL1 | Zinc Finger Protein, Pleiomorphic Adenoma | 5325 |
| | | Gene-Like 1 | |
| 51. | PON1 | Paraoxonase 1 | 5444 |
| 52. | PON2 | Paraxonase 2 | 5445 |
| 53. | PON3 | Paraoxonase 3 | 5446 |
| 54. | PPP1R9A | Protein Phosphatase Inhibitor, Protein | 55607 |
| | | Phosphatase 1, Regulatory (Inhibitor) Subunit 9A | |
| 55. | RASGRF1 | Guanine Nucleotide Exchange Factor, Ras | 5923 |
| | | Protein-Specific Guanine Nucleotide-Releasing | |
| | | Factor 1 | |
| 56. | SANG | GNAS1 Antisense | 149775 |
| 57. | SDHD | Succinate Dehydrogenase, Subunit, Succinate | 6392 |
| | | Dehydrogenase Complex, Subunit D, Integral | |
| | | Membrane Protein | |
| 58. | SGCE | Sarcoglycan, Epsilon | 8910 |
| 59. | SLC22A18 | Organic Cation Transporter, Solute Carrier | 5002 |
| | | Family 22 (Organic Cation Transporter), Member | |
| | | 18 | |
| 60. | SLC22A2 | Organic Cation Transporter, Solute Carrier | 6582 |
| | | Family 22 (Organic Cation Transporter), Member 2 | |
| 61. | SLC22A3 | Solute Carrier Family 22 (Extraneuronal | 6581 |
| | | Monoamine Transporter), Member 3 | |
| 62. | SLC38A4 | Amino Acid Transporter, Solute Carrier Family | 55089 |
| | | 38, Member 4 | |
| 63. | SNURF- | SNRPN Upstream Reading Frame, Hypothetical | 6638 |
| | SNRPN | Protein LOC145622 | |
| 64. | TCEB3C | Transcription Elongation Factor, Transcription | 162699 |
| | | Elongation Factor B Polypeptide 3C (Elongin | |
| | | A3) | |
| 65. | TRPM5 | PD NI P Ca2+-Activated Cation Channel, | 29850 |
| | | Transient Receptor Potential Cation Channel, | |
| | | Subfamily M, Member 5 | |
| 66. | TSIX | X (Inactive)-Specific Transcript, Antisense | 9383 |
| 67. | TSSC4 | Tumor Suppressing Candidate, Tumor | 10078 |
| | | Suppressing Subtransferable Candidate 4 | |
| 68. | UBE3A | Ubiquitin Protein Ligase, Ubiquitin Protein | 7337 |
| | | Ligase E3A (Human Papilloma Virus E6- | |
| | | Associated Protein, Angelman Syndrome) | |
| 69. | USP29 | Ubiquitin-Specific Protease, Ubiquitin Specific | 57663 |
| | | Peptidase 29 | |
| 70. | WT1 | Zinc Finger Protein, Wilms Tumor 1 | 7490 |
| 71. | ZIM2 | Zinc-Finger Protein, Zinc Finger, Imprinted 2 | 23619 |
| 72. | ZIM3 | Zinc-Finger Protein (Human Zinc Finger, | 114026 |
| | | Imprinted 3) | |
| 73. | ZNF127AS | Zinc Finger Protein 127 Antisense | 10108 |
| 74. | ZNF215 | Zinc Finger Protein, Zinc Finger Protein 215 | 7762 |
| 75. | ZNF264 | Zinc-Finger Protein (Human Zinc Finger Protein | 9422 |
| | | 264) | |

TABLE 7
A list of candidate genes which can get methylated

| Sl. | Gene | |
|---|---|---|
| No. | Symbol | Gene Description |
| 1. | APAF1 | Apoptotic Protease Activating Factor |
| 2. | ARPC1B | Actin Related Protein 2/3 Complex, Subunit 1 B, |
| | | 41 kda |
| 3. | BIRC5 | Baculoviral IAP Repeat-Containing 5 (Survivin) |
| 4. | BRCA1 | Breast Cancer 1, Early Onset |
| 5. | CASP8 | Caspase 8, Apoptosis-Related Cysteine Protease |
| 6. | CD44 | CD44 Antigen (Homing Function And Indian |
| | | Blood Group System) |
| 7. | CDH1 | Cadherin 1, Type 1, E-Cadherin (Epithelial) |
| 8. | CDH13 | Cadherin 13, H-Cadherin (Heart) |
| 9. | CDKN2A | Cyclin-Dependent Kinase Inhibitor 2A (Melanoma, |
| | | P16, Inhibits CDK4) |
| 10. | CEBPA | CCAAT/Enhancer Binding Protein (C/EBP), Alpha |
| 11. | DAPK1 | Death-Associated Protein Kinase 1 |
| 12. | DDX53 | DEAD (Asp-Glu-Ala-Asp) Box Polypeptide 53 |
| 13. | ESR1 | Estrogen Receptor 1 |
| 14. | FAS | Fas (TNF Receptor Superfamily, Member 6) |
| 15. | FHIT | Fragile Histidine Triad Gene |
| 16. | GJB1 | Gap Junction Protein, Beta 1, 32 kda (Connexin 32, |
| | | Charcot-Marie-Tooth Neuropathy, X-Linked) |
| 17. | MGMT | O-6-Methylguanine-DNA Methyltransferase |
| 18. | MST1 | Macrophage Stimulating 1 (Hepatocyte Growth |
| | | Factor-Like) |
| 19. | MSTP9 | Macrophage Stimulating, Pseudogene 9 |
| 20. | MYCN | V-Myc Myelocytomatosis Viral Related Oncogene, |
| | | Neuroblastoma Derived (Avian) |
| 21. | NANOG | Nanog homeobox |
| 22. | PGR | Progesterone Receptor |
| 23. | POU2AF1 | POU Domain, Class 2, Associating Factor 1 |
| 24. | Oct4 | POU domain, Class 5, transcription factor 1 |
| 25. | PTEN | Phosphatase And Tensin Homolog (Mutated In |
| | | Multiple Advanced Cancers 1) |
| 26. | RASSF1 | Ras Association (Ralgds/AF-6) Domain Family 1 |
| 27. | SFN | Stratifin |
| 28. | SFRP1 | Secreted Frizzled-Related Protein 1 |
| 29. | THBS1 | Thrombospondin 1 |
| 30. | TIMP3 | Tissue Inhibitor Of Metalloproteinase 3 (Sorsby |
| | | Fundus Dystrophy, Pseudoinflammatory) |
| 31. | TNFRSF10C | Tumor Necrosis Factor Receptor Superfamily, |
| | | Member 10c, Decoy without An Intracellular |
| | | Domain |
| 32. | TP53 | Tumor Protein P53 (Li-Fraumeni Syndrome) |
| 33. | TGFB2 | Transforming Growth Factor, Beta 2 |

The invention is further elaborated with the help of following examples. However, these examples should not be construed to limit the scope of the invention.

### EXAMPLES

### Example 1

Endotoxin induced silencing of mesoderm induction and functional differentiation: Role of the DNA-binding cytokine HMGB1 in pluripotency and infection. This example is explained with the help of following sub-examples:

### Example 1(i)

Culture of hESCs & production of EBs: Human embryonic stem cell line HUES-9 was obtained from Harvard University and was used after institutional ethics committee approval. They were maintained on mouse embryonic feeder (MEF) cells. HUES-9 was maintained in embryonic stem cell medium (ES medium) consisting of 80% KnockOut DMEM and 20% KnockOut serum replacement (KSR), supplemented with 2 mM L-glutamine, 1% non-essential amino acid solution, 0.1 mM β-mercaptoethanol, 4 ng/ml human recombinant basic fibroblast growth factor (βFGF), and Penicillin-Streptomycin 50 U/ml (all from Invitrogen, Carlsbad, Calif.). For induction of embryoid body (EB) formation, the hESCs were seeded on low-adherent 60 mm plate (BD Biosciences, San Jose, Calif.) containing ES media without FGF2.

### Example 1 (ii)

Exposure of EBs to LPS: EBs at day-2.5 were exposed to 12.5 pg/ml of Endotoxin/lipopolysaccharide (LPS) (Sigma) for 48 hrs supplemented in culture medium. The normal and the endotoxin treated EBs were harvested on day 4.5. Post exposure, the control and endotoxin treated EBs were divided in two groups. One group was lysed in TRIZOL for RNA isolation and the other group was fixed in 4% paraformaldehyde for immunofluorescence. The expression profile of the ectoderm, endoderm, mesoderm and trophectoderm lineage markers like PHI-tubulin, GATA4, BMP2, Brachury and β-hCG etc. were studied by RT-PCR and Immunofluorescence. The expression of the LPS-inducible and pluripotency related DNA binding protein HMGB1 was also studied in both the control and treated EBs.

### Example 1 (iii)

RT-PCR: Total RNA from cells was isolated using TRIZOL-LS Reagent (Invitrogen) as per the manufacturer's protocol. Complementary DNA was synthesized using the SuperScript III First-Strand Synthesis System (Invitrogen) as per the manufacturer's instructions. Polymerase Chain Reaction (PCR) was carried out using 1U Tag DNA Polymerase (Sigma) and MgCl₂ to a final concentration of 1.5 mM in a total volume of 25 μl/reaction. β-actin and GAPDH were used as the housekeeping controls. PCR cycles consisted of an initial denaturation at 95° C. for 5 minutes followed by 35 amplification cycles of denaturation at 94° C. for 45 seconds, annealing for 45 seconds, and extension at 72° C. for 45 seconds and final extension at 72° C. for 10 minutes. The RT-PCR primers, amplicon sizes, and their annealing temperatures are given in Table-8.

TABLE 8
List of genes and RT-PCR primers used

| | | Anneal- | |
|---|---|---|---|
| | | ing | |
| | | Temper- | Product |
| | | ature | Size |
| Gene | Sequence | (° C.) | (bp) |
| Oct 4 | CGACCATCTGCCGCTTTGAG | 57 | 572 |
| | CCCCCTGTCCCCCATTCCTA | | |
| Nanog | CCTCCTCCATGGATCTGCTTATTCA | 57 | 262 |
| | CAGGTCTTCACCTGTTTGTAG | | |
| GAPDH | GGGCGCCTGGTCACCAGGGCTG | 60 | 531 |
| | GGGGCCATCCACAGTCTTCTG | | |
| HMGB1 | GCAGATGACAAGCAGCCTTA | 60 | 104 |
| | TTTGCTGCATCAGGCTTTCC | | |
| βIII | CTTGGGGCCCTGGGCCTCCGA | 60 | 174 |
| Tubulin | GGCTTCCTGCAGTGGTACACGGGCG | | |
| GATA4 | TCCAAACCAGAAAACGGAAG | 60 | 187 |
| | CTGTGCCCGTAGTGAGATGA | | |
| Brachury | ACCCAGTTCATAGCGGTGAC | 60 | 216 |
| | ATGAGGATTTGCAGGTGGAC | | |
| BMP2 | TGTATCGCAGGCACTCAGGTCAG | 60 | 328 |
| | AAGTCTGGTCACGGGGAAT | | |
| hANP | GAACCAGAGGGGAGAGACAGAG | 60 | 383 |
| | CCCTCAGCTTGCTTTTTAGGAG | | |
| cTNT | GGCAGCGGAAGAGGATGCTGAA | 65 | 151 |
| | GAGGCACCAAGTTGGGCATGAACGA | | |
| ABCG2 | GTTTATCCGTGGTGTGTCTGG | 55 | 652 |
| | CTGAGCTATAGAGGCCTGGG | | |
| GATA2 | TGACTTCTCCTGCATGCACT | 60 | 244 |
| | AGCCGGCACCTGTTGTGCAA | | |
| HAND1 | TGCCTCAGAAAGAGAACCAG | 60 | 274 |
| | ATGGCAGGATGAACAAACAC | | |
| BMP4 | GTCCTGCTAGGAGGCGCGAG | 60 | 339 |
| | GTTCTCCAGATGTTCTTTCG | | |
| βhCG | GCTACTGCCCCACCATGACC | 55 | 95 |
| | ATGGACTCGAAGCGCACATC | | |

### Example 1(iv)

Immunofluorescence and Cell counting: HESCs were grown on coverslips coated with MEFs and then fixed with 4% paraformaldehyde and 5% sucrose (Sigma) followed by permeabilization in 0.2% Triton X100 (Sigma). The slides were then incubated with primary antibodies 1:500 dilution of SSEA4 (Chemicon, Calif., USA), 5 ug/ml Nanog (Santa Cruz Biotechnology, CA, USA), 10 μg/ml Brachury (R&D Systems Inc. Minneapolis, USA), and 1.5 μg/ml HMGB1 (Sigma) overnight at 4° C. After washing thrice with PBS, fluorescein isothiocyanate/Texas red-labeled Secondary antibodies against the primary goat/rabbit/mouse were added as 1:500 dilutions and incubated for 2 hours. DAPI (Sigma) was used for nuclear staining and then washed with PBS. The negative controls were done without primary antibodies. Slides were mounted with DABCC (Sigma) and images were acquired using Nikon Eclipse 90i microscope (Nikon Corporation, Japan) and Image-Pro Express software (Media Cybernetics, Inc, Silver Spring, Md.). The results were then compared with the control cells (non-LPS treated EBs). To count the number of cells per EB, the number of DAPI stained nuclei were counted in 10 each of the control and LPS treated EBs.

### Example 1 (v)

Osteoblast differentiation: To assess the differentiating potential of EBs towards tissues of mesoderm origin, embryoid bodies were produced and exposed to LPS as described above. The normal and LPS treated EBs, 30 each, were subjected to osteoblast differentiation from day 5.5 onwards [16]. To stimulate differentiation into osteogenic cells, ES medium containing 10⁻⁸ M Dexamethasone, 50 μg/ml L-ascorbic acid and 5 mM Sodium-beta-glycophosphate was used. The medium was changed every 2-3 days and the differentiation was continued upto 15 days. The osteoblast differentiation was characterized by identifying mineralized areas using von Kossa and Alizarin Red staining [17]. These were visualized and acquired using a Nikon Eclipse 90i microscope (Nikon Corporation, Japan).

### Example 1 (vi)

Comet assay: Detection of DNA damage in individual EBs was carried out with a slight modification of the method described by [18]. Comet tail length was calculated by measuring the streak of DNA comet tail between the edge of the embryoid bodies till the end of tail using Nikon Eclipse 90i microscope (Nikon Corporation, Japan) and Image-Pro Express software (Media Cybernetics, Inc, Silver Spring, Md.).

### Example 1 (vii)

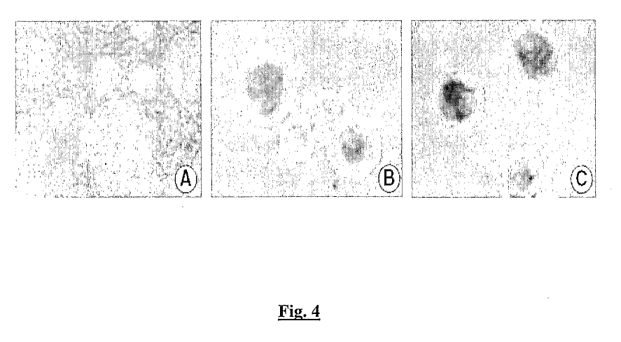

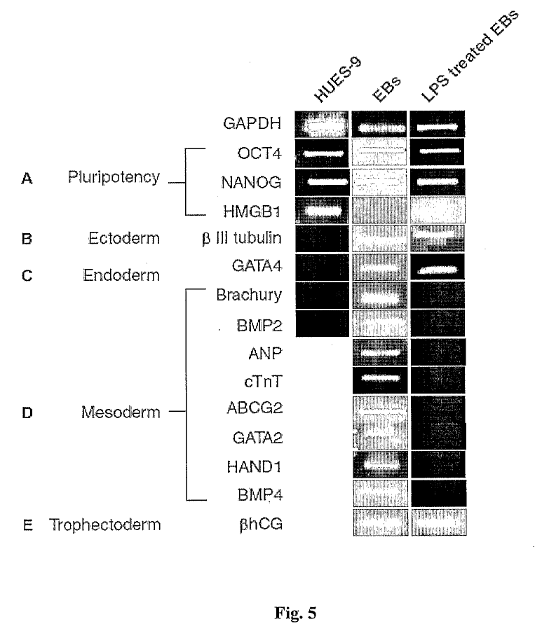

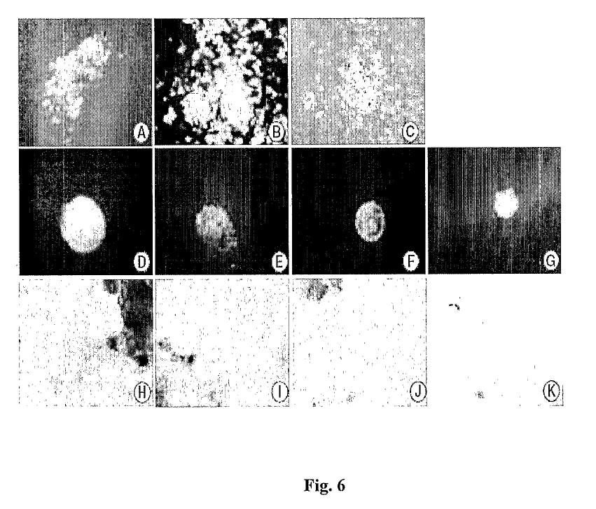

Effect of LPS on the expression of pluripotency, germ lineages markers and HMGB1 in the EBs: In this study we have used five day old embryoid bodies (EBs) as entities equivalent to peri-implantation stage blastocysts. The effect of endotoxins/LPS on the development and induction of lineages in the EBs were examined. The hESC line HUES9 was grown and passaged after every 5 days. FIG. 4A shows normal phase contrast pictures of this cell line grown on supporting MEF cells. The pluripotency of these cells were checked by RT-PCR analysis for Oct4, SSEA4, and Nanog (FIG. 5). The expression of HMGB1 was also confirmed by RT-PCR (FIG. 5). We found positive mRNA expression for Oct4, Nanog, SSEA4 and HMGB1 in these normal hESCs at day-5. The localization and expression of SSEA4, Nanog, and HMGB1 was confirmed by Immunofluorescence (FIG. 6 A, B, and C). SSEA4 was found to be surface localized, where as Nanog, and HMGB1 were localized in the nucleus of the normal HUES9 cells. The mRNA expression of ectoderm, endoderm, mesoderm and trophectoderm lineage markers like βIII-tubulin, GATA4, BMP2, Brachury, and β-hCG were found to be negative in the HUES9 cells, indicating their undifferentiated status.

The HUES9 cells were harvested on day 5 and used for induction of EBs. The control EBs were collected on day 4.5 of culture (FIG. 4b). The LPS treated EBs were also collected on day 4.5 and were compared with the normal for morphological changes under the microscope (FIG. 4c). The normal and LPS treated EBs did not show any visible morphological differences in terms of their shape, size or numbers. The normal and LPS treated EBs were screened for pluripotency and the germ lineage markers. Positive expression for ectoderm, endoderm, mesoderm and trophectoderm lineages markers βIII-tubulin, GATA4, BMP2, Brachury and f3-hCG were found in all the normal EBs on day-4.5 (FIG. 5). HMGB1 mRNA expression was not found in the normal EBs (FIG. 5). The LPS treated EBs showed positive mRNA expression for HMGB1, βIII-tubulin, GATA4 and β-hCG (FIG. 5). We found no mRNA expressions for the two mesoderm markers Brachury and BMP2 in these treated EBs. Inhibition of mesoderm induction was further confirmed by the absence of mRNA expressions for six other mesoderm markers like BMP4, GATA2, ABCG2, cTnT, hANP and HAND1 (FIG. 5). The LPS treated EBs also showed a positive or induced expression of HMGB1 (FIG. 5). Immunofluorescence localization of HMGB1 and the mesoderm marker Brachury was done to check the protein expressions. The normal EBs were positive for Brachury and lacked signals for HMGB1 (FIGS. 6 D and F). The LPS treated EBs showed cytoplasmic localization of HMGB1 and showed no signals for Brachury (FIGS. 6E and G). These EBs, when further tested for Brachury expression on day 9.5, did not show a positive signal. This indicates that the expression of Brachury was actually silenced or downregulated and not merely delayed by LPS.

### Example 1 (viii)

Effect of LPS on differentiation of EBs to Osteoblasts: We found that the normal EBs could be successfully differentiated to osteoblast cells which were characterized by mineral depositions confirmed by Alizarin Red and von Kossa staining at the end of 15 days of differentiation. The normal EBs could be successfully differentiated as evidenced by positive staining for Alizarin Red and von Kossa (FIGS. 6H & J). The LPS treated EBs failed to differentiate into functional osteoblast as indicated by the absence of mineral depositions with no positive signals for Alizarin Red and von Kossa (FIGS. 6L and K).

### Example 1 (ix)

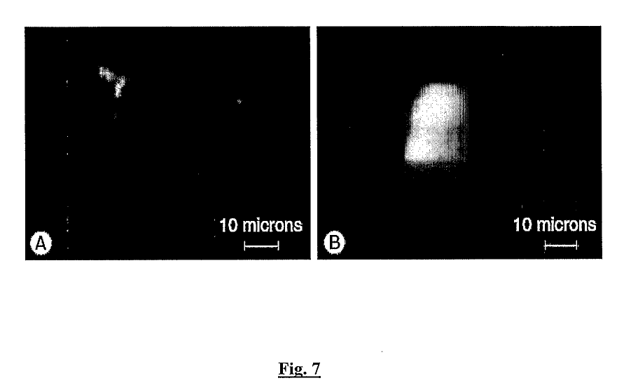

Cell numbers and DNA fragmentation: For a count of the average number of cells per EB, the DAPI stained nuclei were counted in individual control and LPS treated EBs under epifluorescence. The average number of cells/EB (as mean±SD) in the control were 142.33±48.41 cells/EB, and in the LPS treated group were 175±75.47 cells/EB. These values did not differ significantly (P=0.57) as analyzed by a Students t-Test. The LPS treated EBs however showed more DNA tailing or fragmentation (21.48±12.443 μm average) as compared to the control EBs with an average tailing of 2.48±1.0701 μm (FIG. 7).

### Example 2

### Changes in Expression Profiles in Spherical Cavitating/Cystic or Non-Cavitating EBs after Treatment with Various Compounds or Biologicals

Examples of changes in the expression profiles/patterns of lineage markers, pluripotency markers, epigenetic markers and imprinted genes, in day 4.5 spherical cavitating/cytic embryoid bodies upon exposure to compounds like lipopolysaccharide (LPS), Rho kinase inhibitor (Y27632), Azacytidine (Aza), and biologicals like an Uvomorulin antibody (UVO) are given in the table nos. 9-12 below. The sign ‘+’ indicates expression of the gene and the sign “−” indicates non-expression of the gene.

TABLE 9
LPS DAY 4.5 EBs

| GENES | HUES 9 EB D4.5 Control | HUES 9 EB D4.5 LPS |
|---|---|---|
| Brachury | + | − |
| ABCG2 | + | − |
| GATA2 | + | − |
| BMP4 | + | − |
| HAND1 | + | − |
| hANP | + | − |
| cTNT | + | − |
| Dlk1 | + | − |
| SDHD | + | − |
| HMGB1 | − | + |
| DPYS | − | + |
| CDH1 | + | − |

TABLE 10
EFFECT OF UVO TREATMENT

| | HUES 7 EB DAY 4.5 | HUES 7 EB DAY 4.5 |
|---|---|---|
| GENES | CONTROL | UVO |
| BMP-2 | + | − |
| BRACHURY | + | − |
| XIST | − | + |
| HMGB1 | − | + |
| DPYS | − | + |
| CDH1 | − | + |
| TP73 | − | + |
| P53 | + | − |
| TGF-β2 | − | + |
| OCT_4 | + | + |
| CDX2 | − | − |
| β-III TUBULIN | + | + |
| HOXD 12 | − | − |
| CD44 | + | + |
| H 19 | + | + |
| H TERT | − | − |
| HOXD 11 | − | − |
| PTEN | + | + |

TABLE 11
EFFECT OF Y27632 TREATMENT DAY 4.5 EBs

| | HUES 9 EB DAY 4.5 | HUES 9 |
|---|---|---|
| GENES | CONTROL | EB DAY 4.5 RI |
| OCT-4 | − | + |
| CDX-2 | + | − |
| β-III TUBULIN | − | + |
| BRACHURY | + | − |
| DLK-1 | + | − |
| SDHD | + | − |
| HOXD12 | + | − |
| TIMP3 | + | − |
| CD44 | − | + |
| hTERT | + | − |
| PGR | − | + |
| TP73 | − | + |
| NOTCH | − | + |
| p53 | + | − |
| PTEN | − | + |
| TGF β | − | + |
| CDH1 | + | + |
| HOXD11 | + | + |
| BMP-2 | − | − |

TABLE 12
EFFECT OF AZA TREATMENT - EB D 4.5

| | HUES 9 EB D4.5 | HUES 9 |
|---|---|---|
| GENES | CONTROL | EB D 4.5 AZA |
| OCT-4 | − | + |
| NANOG | + | − |
| CDX2 | + | − |
| β-III TUBULIN | − | − |
| BMP-2 | − | − |
| BRACHURY | + | − |
| DLK1 | + | − |
| SDHD | + | − |
| HOXD12 | + | − |
| CDH13 | + | − |
| TIMP3 | + | − |
| TNFR | + | − |
| CD44 | − | − |
| H19 | − | − |
| DPYS | − | + |
| CDH1 | + | − |
| hTERT | + | − |
| p53 | + | − |
| HOXD12 | + | − |
| PTEN | − | − |

### Example 3

### Our In Vitro Model can be Used to Detect Both the Positive and the Negative Effects of a Molecule on the Implantation of an Embryo

Example showing a positive effect of a compound on implantation.

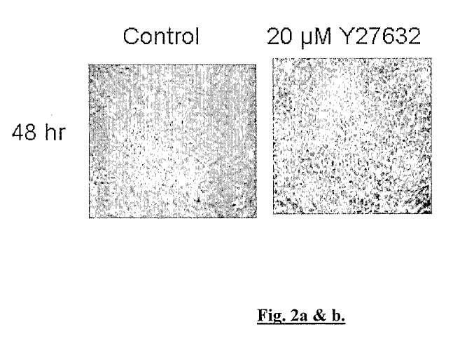

A supportive/enhanced attachment of Spheroid cavitating/cytic EB was seen in our implantation model as a result of 20 μM Y27632 exposure (FIG. 2).

Method: Spherical Cytic EBs were collected on day 4.5 and exposed to the continuous presence of Y27632 (various doses were used). After 48 hrs of exposure with 20 μM Y27632 the embryo like entity has attached and outgrown to a larger area. The control cytic EB had however just attached with smaller area of outgrowth showing that this dose of Y27632 can help in initial implantation of embryo during pregnancy.

### Negative Effect as Determined Using Our Model:

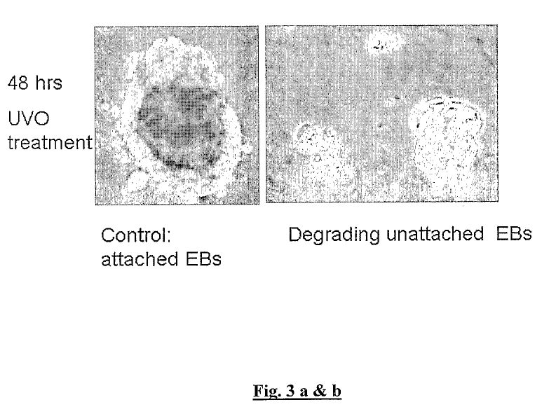

A high dose of compounds like LPS/Azacytidine/DMSO etc. or biologicals like uvomorulin antibody for 48 hrs caused degeneration of the EBs, and they failed to attach and outgrow, indicating a detrimental effect of these compounds in the initial days of pregnancy (FIG. 3).

### Example 4

### Methylation specific PCRs (MSPs)

Methylation status of several of the epigenetically regulated genes and the imprinted genes in response to exposure to various compounds/drugs or biologicals were screened.

### Example 4(a)

Normal or control EBs vs. LPS (48 hrs, 5 ug/ml) treated EBs showed positive mRNA expression of SDHD, DYPS and CDH1. MSP analysis after bisulphite treatment of the DNA showed that one of the alleles were methylated, with positive bands for both the modified and unmodified primers.

### Example 4(b)

After the EBs were exposed to LPS: Showed negative mRNA expression/silencing of SDHD, DYPS and CDH1. MSP analysis after bisulphite treatment of the DNA showed that both the alleles of these genes were methylated, with positive bands for the unmodified primers only, indicating that the toxin causes a silencing of genes by hypermethylation of their promoter.

### REFERENCES

1. Thomson J A, Itskovitz-Eldor J, Shapiro S S, et al. Embryonic stem cell lines derived from human blastocysts. Science 282:1145-1147 (1998).
2. Reubinoff B E, Pera M F, Fong C Y, et al. Embryonic stem cell lines from human blastocysts: somatic differentiation in vitro. Nat. Biotechnol. 18: 399-404 (2000).
3. Deb K D, Devi Prakash A, Sharma V, Totey S. Embryonic Stem Cells: From Markers to Market. Rejuvenation Res. (accepted) (2007)
4. Carpenedo R L, Sargent C Y, McDevitt T C. Rotary Suspension Culture Enhances the Efficiency, Yield and Homogeneity of Embryoid Body Differentiation. Stem Cells (2007) (In Press).
5. Dvash T, Benvenisty N. Human embryonic stem cells as a model for early human development. Best Pract Res Clin Obstet Gynaecol; 18 (6):929-940 (2004)
6. Deb K, Chatturvedi M M and Jaiswal Y K. Gram-negative bacterial endotoxin induced infertility: A bird's eye view. Gynecol Obstet. Invest. 57:224-232 (2004).
7. Romero R, Espinoza J, Mazor. Can endometrial infection/inflammation explain implantation failure, spontaneous abortion, and preterm birth after in vitro fertilization? Fertility and Sterility 82(4): 799-80 (2004).
8. Deb K, Chaturvedi M M, Jaiswal Y K. Comprehending the role of LPS in Gram-negative Bacterial Vaginosis: Ogling into the Causes of Unfulfilled Child-wish. Arch Gynecol Obstet. 270(3): 133-146 (2004).
9. Deb K, Chaturvedi M M, Jaiswal Y K. A “minimum dose” of LPS required for implantation failure: assessment of its effect on the maternal reproductive organs and IL-1α expression in mouse. Reproduction. 128 (1):87-97 (2004).
10. Deb K, Chaturvedi M M and Jaiswal Y K. The role of TNF-α in Gram-negative bacterial LPS induced Implantation failure. Reprod Med. Biol. 4: 79-88 (2005).
11. Deb K, Chaturvedi M M, Jaiswal Y K. Gram-negative bacterial LPS induced poor uterine receptivity and implantation failure in mouse: alterations in IL-1β expression in the preimplantation embryo and uterine horns. Infect Dis Obstet Gynecol. 13 (3): 125-133 (2005)
12. Yang H, Wang H, Czura C J, and Tracy K J. The cytokine activity of HMGB1. J of Leukoc. Biol. 78; 1-8. (2005)
13. Skottman H, Mikkola M, Lundin K et al. Gene Expression Signatures of Seven Individual Human Embryonic Stem Cell Lines. Stem cells. 23:1343-1356 (2005).
14. Adjaye J, Huntriss J, Herwig R et al. Primary Differentiation in the Human Blastocyst: Comparative Molecular Portraits of Inner Cell Mass and Trophectoderm Cells. Stem Cells. 23(10): 1514-1525 (2005)
15. Jaiswal Y K, Chaturvedi M M, Deb K. Effect of bacterial endotoxins on superovulated mouse embryos in vivo: Is CSF-1 involved in endotoxin induced pregnancy loss? Infect Dis Obstet. Gynecol. 32050:1-9 (2006).
16. Duplomb L, Dagouassat M, Jourdon P et al. Embryonic stem cells: new tool to study osteoblast and osteoclast differentiation. Stem Cells. (2006) (EPub)
17. Karp J M, Ferraeira L S, Ali K et al. Cultivation of Human Embryonic stem cells without the embryoid body step enhances ostogenesis in vitro. Stem cells 24:835-843 (2006).
18. Takahashi M, Saka N, Takahashi H et al. Assessment of DNA Damage in Individual Hamster Embryos by Comet Assay. Molecular Reproduction and Development 54:1-7 (1999).
19. Edwards R G, Ludwig M. Are major defects in children conceived in vitro due to innate problems in patients or to induced genetic damage? Reprod Bio Med. 7(2):131-138 (2003).
20. Ingalls R R, Rice A P, Qureshi N et al. The Inflammatory Cytokine Response to Chlamydia trachomatis Infection Is Endotoxin Mediated. Infection and Immunity 3125-3130 (1995).
21. Rinehart J J and Keville L. Effects of endotoxin on proliferation of human hematopoietic cell precursors. Cytotechnology 24 (2):153-159 (1997).
22. Hyslop L A, Armstrong L, Stojkovic M, Lako M. Human embryonic stem cells: biology and clinical implications. Expert Rev Mol. Med. 7(19):1-21 (2005).
23. Adler S., Paparella M., Pellizzer C., Hartung T. and Bremer S. (2005) The Detection of Differentiation-inducing Chemicals by using Green Fluorescent Protein expression in Genetically Engineered Teratocarcinoma Cells. Alternatives to laboratory animals: Altern Lab Anim 33, 1-13.
24. Adler S., Pellizzer C., Paparella M., Hartung T., Bremer S. (2005) The Effects of Solvents on Embryonic Stem Cell Differentiation. Toxicology in vitro, in press.
25. Adler S., Pellizzer C., Hareng L., Hartung T., Bremer S. (2005) Requirements for the establishment of an in vitro model for developmental toxicity testing based on human embryonic stem cells. Toxicol Appl Pharmacol, submitted.
26. Bremer S., Pellizzer C., Adler S., Paparella M. and de Lange J. (2002) Development of a testing strategy for detecting embryotoxic hazards of chemicals in vitro by using embryonic stem cell models. Altern Lab Anim 27, 107-109.
27. Pellizzer C., Adler S., Corvi R., Hartung T. and Bremer S. (2004a) Monitoring of teratogenic effects in vitro by analysing a selected gene expression pattern. Toxicol In Vitro 3, 325-335.
28. Pellizzer C., Bello E., Adler S., Hartung T. and Bremer S. (2004b) Detection of tissue-specific effects by methotrexate on differentiating mouse embryonic stem cells. Birth Defects Res B Dev Reprod Toxicol 5, 331-341.
29. Jaiswal Y K, Jaiswal M K, Agrawal V, Deb K (2007) Maternal Gram-negative bacterial infection induced apoptosis of the implanting blastocyst. Journal of Turkish-German Gynecology Association. 7 (4).
30. Nonylphenol and Octylphenol-Induced Apoptosis in Human Embryonic Stem Cells is Related to Fas-Fas Ligand Pathway (2006) Kim S, Kim B, Shim J, Gil J, Yoon Y, Kim J. Toxicological Sciences, doi:10.1093/toxsci/kf1114.
31. Mosmann T. Rapid colorimetric assay for cellular growth and survival: application to proliferation and cytotoxicity assays. J Immunol Methods. 1983 Dec. 16; 65(1-2):55-63.
32. Bader, A., Al Dubai, H., and Weitzer, G. (2000). Leukemia inhibitory factor modulates cardiogenesis in embryoid bodies in opposite fashions. Circ. Res. 86, 787-794.
33. Buttery, L. D., Bourne, S., Xynos, J. D., Wood, H., Hughes, F. J., Hughes, S. P., Episkopou, V., and Polak, J. M. (2001). Differentiation of osteoblasts and in vitro bone formation from murine embryonic stem cells. Tissue Eng. 7, 89-99.
34. Lumelsky, N., Blondel, O., Laeng, P., Velasco, I., Ravin, R., and McKay, R. (2001). Differentiation of Embryonic Stem Cells to Insulin-Secreting Structures Similiar to Pancreatic Islets. Science. 292, 1389-1394.
35. Lee, S. H., Lumelsky, N., Studer, L., Auerbach, J. M., and McKay, R. D. (2000).
36. Efficient generation of midbrain and hindbrain neurons from mouse embryonic stem cells. Nat. Biotechnol. 18, 675-679.
37. Schuldiner M, Eiges R, Eden A, Yanuka O, Itskovitz-Eldor J, Goldestien R S, Benvenisty N (2001) Induced neuronal differentiation of human embryonic stem cells. Brain Research. 913, 201-205.

1) An in vitro embryonic model comprising spherical smooth-embryoid body (SSE) for determining effect of molecule based on expression of gene HMGB1.

2) The in vitro embryonic model as claimed in claim 1, wherein said model identifies stage of the SSE development at which the molecule acts.

3) The in vitro embryonic model as claimed in claim 1, wherein said SSE is about 3-6 days old.

4) The in vitro embryonic model as claimed in claim 1, wherein said SSE is about 100-400 μm in diameter.

5) The in vitro embryonic model as claimed in claim 1, wherein said SSE is obtained from stem cells selected from a group comprising embryonic stem cells (ESCs), embryonic germ cells (EGCs) and embryonic carcinoma cells (ECCs).

6) The in vitro embryonic model as claimed in claim 1, wherein said effect is selected from a group comprising sternness, pluripotency, embryotoxicity, development defects, lineage induction, formation of tissues, arrested growth, cell proliferation, epigenetic changes, chromosomal aberrations, karyotypic changes, cytotoxicity, cell migration, interaction with extracellular matrix components, effect on niche components of cells, mutagenesis, pharmacogenetic effects and toxicogenetic effects.

7) The in vitro embryonic model as claimed in claim 1, wherein said molecule is selected from a group comprising drugs, formulations, contraceptives, herbal extract or preparation, environment pollutants, endotoxins, nanoparticles, viruses, microbial toxins, biologicals, antibodies, proteins, DNA, RNA and siRNAs.

8) The in vitro embryonic model as claimed in claim 1, wherein the molecule is lipopolysaccharide.

9) An in vitro method for determining effect of molecule on spherical smooth-embryoid body (SSE) comprising acts of:
a) exposing the molecule to the SSE, and
b) screening the SSE for expression of gene HMGB1, the effect of the exposure on germ lineages, implantating embryo and on differentiation into tissue;

10) The method as claimed in claim 9, wherein said method is carried out using the in vitro embryonic model.

11) The method as claimed in claim 9, wherein said molecule is selected from a group comprising drugs, formulations, contraceptives, herbal extract or preparation, environment pollutants, endotoxins, nanoparticles, viruses, microbial toxins, biologicals, antibodies, proteins, DNA, RNA and siRNAs.

12) The method as claimed in claim 9, wherein the molecule is lipopolysaccharide.

13) The method as claimed in claim 9, wherein said screening is carried out by studying expression of markers selected from a group comprising lineage markers, pluripotency markers and epigenetic markers or any combination(s) thereof.

14) The method as claimed in claim 13, wherein said lineage markers are selected from a group comprising ectoderm markers, endoderm markers, mesoderm markers and trophectoderm markers.

15) The method as claimed in claim 13, wherein said epigenetic markers are selected from a group comprising imprinted genes and candidate genes which can get methylated.

16) The method as claimed in claim 13, wherein the expression of marker is studied by using techniques selected from a group comprising RT-PCR, flow cytometry and immunofluorescence.

17) An in vitro embryo implantation model comprising:
a) coat of extracellular matrix onto support matrix having well(s) or cavity;
b) layer of endometrial cells onto the extracellular matrix; and
c) spherical smooth-embryoid body (SSE) placed into the well or cavity to determine effect of molecule based on expression of gene HMGB1.

18) The in vitro embryo implantation model as claimed in claim 17, wherein said model identifies stage of the SSE development at which the molecule acts.

19) The in vitro embryo implantation model as claimed in claim 17, wherein said extracellular matrix is selected from a group comprising fibronectin, collagen, matrigel, laminin, gelatin, albumin, poly-d-lysine, vitonectin and entactin.

20) The in vitro embryo implantation model as claimed in claim 17, wherein said support matrix is selected from a group comprising agar, low melting agarose, polyacrylamide, gelatin, collagen, chitosan and 3D collagen or polymer scaffolds.

21) The in vitro embryo implantation model as claimed in claim 17, wherein said endometrial cell is selected from a group comprising mouse endometrial cell, human endometrial cell, rabbit endometrial cell, murine endometrial cell, porcine endometrial cell, bovine primary endometrial stromal cell and endometrial stromal cell lines, preferably mouse endometrial stromal cell and human endometrial stromal cell.

22) The in vitro embryo implantation model as claimed in claim 17, wherein said SSE is about 3-6 days old.

23) The in vitro embryo implantation model as claimed in claim 17, wherein said SSE is about 100-400 μm in diameter.

24) The in vitro embryo implantation model as claimed in claim 17, wherein said SSE is obtained from stem cells selected from a group comprising embryonic stem cells (ESCs), embryonic germ cells (EGCs), embryonic carcinoma cells (ECCs).

25) The in vitro embryo implantation model as claimed in claim 17, wherein said effect is selected from a group comprising embryotoxicity, detection of activities of drugs/biologicals which are (a) detrimental to embryonic development and pregnancy, (b) detrimental to lineage induction and tissue formation, (c) inhibit embryo implantation or attachment, (d) inhibit migration and invasion of cells, (e) beneficial for developing embryo, (f) improves attachment of the embryo, (g) improves lineage induction and tissue formation, (h) improves cell proliferation, (i) improves migration and invasion of cells and (j) modulates secretion of growth factors, cytokines and hormones, mutagenesis, pharmacogenetic effects and toxicogenetic effects.

26) The in vitro embryo implantation model as claimed in claim 17, wherein said molecule is selected from a group comprising drugs, formulations, contraceptives, herbal extract or preparation, environment pollutants, endotoxins, nanoparticles, viruses, microbial toxins, biologicals, antibodies, proteins, DNA, RNA and siRNAs.

27) An in vitro method of determining effect of lipopolysaccharide (LPS) on embryoid bodies (EBs), said method comprising acts of:
a. exposing the EBs to the LPS to trigger expression of gene HMGB1 in cytoplasm of the EBs, and
b. observing silencing of mesoderm induction and functional differentiation in the EBs.

28) The in vitro method as claimed in claim 27, wherein said silencing of mesoderm induction and functional differentiation leads to defect in formation of bone, blood and/or heart muscle.

29) The in vitro method as claimed in claim 27, wherein expression of the gene HMGB1 in nucleus of the EBs helps in maintenance of pluripotency in the EBs.


SEQUENCE LISTING (30 sequences)

SEQ ID NO: 1 - DNA, 20 bp, Artificial Sequence
  synthetic construct
cgaccatctgccgctttgag

SEQ ID NO: 2 - DNA, 20 bp, Artificial Sequence
  synthetic construct
ccccctgtcccccattccta

SEQ ID NO: 3 - DNA, 25 bp, Artificial Sequence
  synthetic construct
cctcctccatggatctgcttattca

SEQ ID NO: 4 - DNA, 21 bp, Artificial Sequence
  synthetic construct
caggtcttcacctgtttgtag

SEQ ID NO: 5 - DNA, 22 bp, Artificial Sequence
  synthetic construct
gggcgcctggtcaccagggctg

SEQ ID NO: 6 - DNA, 21 bp, Artificial Sequence
  synthetic construct
ggggccatccacagtcttctg

SEQ ID NO: 7 - DNA, 20 bp, Artificial Sequence
  synthetic construct
gcagatgacaagcagcctta

SEQ ID NO: 8 - DNA, 20 bp, Artificial Sequence
  synthetic construct
tttgctgcatcaggctttcc

SEQ ID NO: 9 - DNA, 21 bp, Artificial Sequence
  synthetic construct
cttggggccctgggcctccga

SEQ ID NO: 10 - DNA, 25 bp, Artificial Sequence
  synthetic construct
ggcttcctgcagtggtacacgggcg

SEQ ID NO: 11 - DNA, 20 bp, Artificial Sequence
  synthetic construct
tccaaaccagaaaacggaag

SEQ ID NO: 12 - DNA, 20 bp, Artificial Sequence
  synthetic construct
ctgtgcccgtagtgagatga

SEQ ID NO: 13 - DNA, 20 bp, Artificial Sequence
  synthetic construct
acccagttcatagcggtgac

SEQ ID NO: 14 - DNA, 20 bp, Artificial Sequence
  synthetic construct
atgaggatttgcaggtggac

SEQ ID NO: 15 - DNA, 23 bp, Artificial Sequence
  synthetic construct
tgtatcgcaggcactcaggtcag

SEQ ID NO: 16 - DNA, 19 bp, Artificial Sequence
  synthetic construct
aagtctggtcacggggaat

SEQ ID NO: 17 - DNA, 22 bp, Artificial Sequence
  synthetic construct
gaaccagaggggagagacagag

SEQ ID NO: 18 - DNA, 22 bp, Artificial Sequence
  synthetic construct
ccctcagcttgctttttaggag

SEQ ID NO: 19 - DNA, 22 bp, Artificial Sequence
  synthetic construct
ggcagcggaagaggatgctgaa

SEQ ID NO: 20 - DNA, 25 bp, Artificial Sequence
  synthetic construct
gaggcaccaagttgggcatgaacga

SEQ ID NO: 21 - DNA, 21 bp, Artificial Sequence
  synthetic construct
gtttatccgtggtgtgtctgg

SEQ ID NO: 22 - DNA, 20 bp, Artificial Sequence
  synthetic construct
ctgagctatagaggcctggg

SEQ ID NO: 23 - DNA, 20 bp, Artificial Sequence
  synthetic construct
tgacttctcctgcatgcact

SEQ ID NO: 24 - DNA, 20 bp, Artificial Sequence
  synthetic construct
agccggcacctgttgtgcaa

SEQ ID NO: 25 - DNA, 20 bp, Artificial Sequence
  synthetic construct
tgcctcagaaagagaaccag

SEQ ID NO: 26 - DNA, 20 bp, Artificial Sequence
  synthetic construct
atggcaggatgaacaaacac

SEQ ID NO: 27 - DNA, 20 bp, Artificial Sequence
  synthetic construct
gtcctgctaggaggcgcgag

SEQ ID NO: 28 - DNA, 20 bp, Artificial Sequence
  synthetic construct
gttctccagatgttctttcg

SEQ ID NO: 29 - DNA, 20 bp, Artificial Sequence
  synthetic construct
gctactgccccaccatgacc

SEQ ID NO: 30 - DNA, 20 bp, Artificial Sequence
  synthetic construct
atggactcgaagcgcacatc